(19) United States
(12) Patent Application Publication
(10) Pub. No.: US 2011/0111026 A1
(43) Pub. Date: May 12, 2011

(54) HUMIDITY-RESISTANT DRUG FORMULATIONS AND METHODS OF PREPARATION THEREOF

(76) Inventors: Adel Penhasi, Holon (IL); Yaakov Stephane Attali, Jerusalem (IL)

(21) Appl. No.: 12/933,843

(22) PCT Filed: Mar. 10, 2009
(86) PCT No.: PCT/IL09/00267

§ 371 Date: Jan. 10, 2011

Related U.S. Application Data
(60) Provisional application No. 61/035,081, filed on Mar. 10, 2008.

(51) Int. Cl. A61K 9/30; A61K 9/32; A61K 9/40; A61K 9/36; A61K 31/40; A61P 9/00; B05D 1/36

(52) U.S. Cl. 424/465; 424/475; 424/482; 424/478; 424/479; 514/423; 427/2.14

(57) ABSTRACT

The invention relates to a pharmaceutical composition of a humidity-sensitive core comprising an active ingredient or pharmaceutically acceptable salt thereof; a coating over the core, the coating containing a cationic polymer; and an additional coating over the cationic polymer-containing coating, with the additional coating including an acidifying agent. Also, methods for preparing such compositions wherein a cationic polymer containing coating is applied over a humidity-sensitive core that contains the active ingredient or pharmaceutically acceptable salt thereof; and then an additional coating is applied over the cationic polymer containing coating.

### FIELD OF THE INVENTION

The present invention provides humidity-resistant pharmaceutical compositions, as well as compositions for protecting sensitive active ingredients from environmental parameters such as oxidation, methods of preparing same; and therapeutic methods utilizing same.

### BACKGROUND OF THE INVENTION

Humidity-sensitive active pharmaceutical ingredients and pharmaceutical formulas have special formulation, process or packaging requirements. Contact of such active ingredients and pharmaceutical formulas with humidity can result in chemical degradation or generation of altered and unwanted polymorphs or isoforms of the active ingredient and/or in alteration of the physico-chemical patterns of the formula (such as appearance, dissolution rate, disintegration time and so forth).

Specialized humidity-resistant packaging is one means for protecting humidity-sensitive active ingredients and formulations. Use of Alu/Alu (Aluminum/Aluminum) blisters, for example (as described, for example, in United States Patent Application Publication Number 2004/0104142) is a technique well-known in the art for protecting such active ingredients and formulations from humidity. However, such methods add cost and complexity to the packaging process.

Use of humidity-resistant coatings is another method for protecting humidity-sensitive active ingredients and formulations. For example, high amounts of coatings that contain a cationic polymer (e.g. Eudragit E™) protect cores from humidity. Such coatings are generally soluble in acidic medium, but (particularly when present in sufficient thickness to confer humidity resistance) do not exhibit rapid, homogeneous release in mildly acidic or neutral medium, and thus are not entirely suitable for active ingredients meant for immediate release. Such properties may affect release rates in vivo, for example if the tablets are rapidly expelled from the stomach into the small intestine, or if the pH of the stomach is higher than usual, e.g. due to the presence of food. Coatings such as Opadry® or Opadry II® or Opadry tm® cannot confer extensive moisture protection even when present in greater than conventional thickness. When present in high amounts, the Opadry coatings also substantially increase lag time for dissolution.

Examples of humidity-sensitive active pharmaceutical ingredients are statins in general and atorvastatin in particular. Atorvastatin of formula [R—R*,R*)]-2-(4-Fluorophenyl)-β,δ-dihydroxy-5-(1-methylethyl)-3-phenyl-4-[(phenylamino)-carbonyl]-1H-pyrrole-1-heptanoic acid and pharmaceutically acceptable salts thereof (see for example U.S. Pat. No. 5,273,995 to Warner-Lambert, hereby incorporated by reference as if fully set forth herein) is a well-known lipid lowering agent. One optional but preferred form of atorvastatin is the pharmaceutically acceptable hemi-calcium salt form, atorvastatin calcium, because it has advantageous stability and bioefficacy. Contact of atorvastatin with humidity can result in generation of altered forms thereof (e.g. atorvastatin lactone).

Atorvastatin is an inhibitor of 3 hydroxy-3-methylglutaryl-coenzyme A (HMG-CoA) reductase. This enzyme catalyzes the conversion of HMG-CoA to mevalonate, an early and rate-limiting step in cholesterol biosynthesis. It is usually administered orally.

Thus, humidity-resistant dosage forms, especially those with rapid and homogeneous release in mildly acidic and neutral medium, and methods for manufacturing same are needed in the art.

### SUMMARY OF THE INVENTION

The present invention overcomes deficiencies of the background art by providing coated formulations, methods of use thereof and methods of manufacture thereof that are humidity-resistant and/or resistant to environmental factors such as oxidation, yet can provide immediate release, comparable to that of the uncoated cores, thus having suitable pharmacokinetics for sensitive active ingredients and/or compositions.

In some embodiments, the active ingredient is humidity sensitive while in other embodiments, the composition overall or a portion thereof is humidity sensitive. In certain embodiments, both the composition overall or a portion thereof and the active ingredient are humidity-sensitive. The humidity sensitive portion of the composition may optionally comprise the core for example. Also alternatively or additionally, the present invention in at least some embodiments protects the core or its active agent from environmental factors such as oxidation, in which case the acidic agent containing coating increases the in vivo dissolution/opening of the cationic polymer coat.

Without wishing to be limited in any way or to provide a closed list, another purpose of the present invention is to prolong the shelf life of a composition containing a humidity-sensitive active ingredient. The principles of the present invention are exemplified hereinbelow for atorvastatin as one non-limiting example.

The present invention provides humidity-resistant pharmaceutical compositions, comprising: a core comprising an active ingredient or pharmaceutically acceptable salt thereof; a cationic polymer containing coating over the core; and another coating over the cationic polymer containing coating, the additional coating comprising an acidifying agent and/or any agent which is able to improve the homogeneity and/or to increase the rate of the dissolution or degradation of the cationic polymer containing coating; methods of preparing same; and therapeutic methods utilizing same. As described herein, the term “cationic polymer containing coating” includes any coating comprising a cationic polymer.

In one embodiment, the present invention provides a pharmaceutical composition, comprising: a core comprising an active ingredient or pharmaceutically acceptable salt thereof; a cationic polymer containing coating over the core and an additional coating over the cationic polymer containing coating, the additional coating comprising an acidifying agent. Preferably, the pharmaceutical composition is a humidity-resistant pharmaceutical composition. Additionally or alternatively, the active ingredient is preferably a humidity-sensitive active ingredient. In another embodiment, another agent (other than an acidifying agent) capable of causing or accelerating dissolution of the inner cationic polymer layer is utilized. Each possibility represents a separate embodiment of the present invention.

In another embodiment, the present invention provides a method for preparation of a pharmaceutical composition comprising an active ingredient or a pharmaceutically acceptable salt thereof, comprising the steps of: (a) applying a cationic polymer containing coating over a core comprising the active ingredient or pharmaceutically acceptable salt thereof; and (b) applying an additional coating over the cationic polymer containing coating, wherein the additional coating comprises an acidifying agent. Preferably, the pharmaceutical composition is a humidity-resistant pharmaceutical composition. Additionally or alternatively, the active ingredient is preferably a humidity-sensitive active ingredient. In another embodiment, the active ingredient is preferentially released in the stomach of a subject. In certain embodiments, the active ingredient is a statin. In another embodiment, the active ingredient is any other active ingredient known in the art. Each possibility represents a separate embodiment of the present invention.

In another embodiment, the present invention provides a method for lowering the cholesterol level of a subject in need thereof, comprising the step of administering to the subject a pharmaceutical composition of the present invention, thereby lowering the cholesterol level of a subject.

In another embodiment, the present invention provides a pharmaceutical composition for lowering the cholesterol level of a subject, comprising a pharmaceutical composition described hereinabove.

In another embodiment, dosage forms of the present invention confer very effective moisture protection without exhibiting a modified dissolution profile relative to uncoated cores, over the entire range of physiological pH conditions, from acidic to neutral. Thus, the compositions of the invention provide the same or at least highly similar dissolution profile and the same or at least highly similar homogeneity compared to the same composition without the cationic coating and the additional coating.

In other embodiments, benefits of the present invention are (a) the possibility of storing the product in bulk quantity (even under ambient temperature and humidity) for a relatively long duration before packaging; (b) ease in the packaging process without requiring particularly low moisture conditions; (c) ability to package the final product in less expensive packaging containers which need not meet very high standards of moisture protection view (e.g. PVC-PVDC blisters or classic plastic bottles, as opposed to e.g. Alu or Aclar® blisters).

In another embodiment, dosage forms of the present invention obviate the need for using a sealing packaging such as an Alu/Alu blister, which is ordinarily a requirement for dosage forms containing a humidity-sensitive active ingredient and/or pharmaceutical composition. Thus, it is possible to use regular packaging materials such as PVC or PVC/PVDC, or Aclar® film, which is polychlorotrifluoroethylene (PCTFE)-based, with dosage forms of the present invention, saving cost and reducing complexity in the packaging process ordinarily associated with Alu/Alu packaging or similar materials.

In another embodiment, dosage forms of the present invention obviate the need for a rapid packaging process after dosage form production, or use of particularly low-moisture conditions or special bulk packaging material for the dosage forms awaiting final packaging. In another embodiment, dosage forms of the present invention obviate the need for dry or reduced room humidity and temperature conditions during the packaging process, enabling packaging to take place under ambient room humidity and temperature.

In another embodiment, dosage forms of the present invention exhibit no significant difference in their release profile of the composition in acidic media (such as gastric fluid or any other acidic media having a pH up to 6.8), compared with existing generic formulations.

Other objects, features and advantages of the present invention will become clear from the following description.

### DETAILED DESCRIPTION OF THE INVENTION

The present invention provides humidity-resistant and/or environmental factor resistant (such as oxidation resistant) pharmaceutical compositions, comprising: a core comprising an active ingredient or pharmaceutically acceptable salt thereof; a cationic polymer containing coating over the core and an additional coating over the cationic polymer containing coating, the additional coating comprising an acidifying agent; methods of preparing same; and therapeutic methods utilizing same. Alternatively or additionally, the present invention in at least some embodiments protects the core or its active agent from humidity and/or oxidation, in which case the acidic agent containing coating increases or accelerates the dissolution/opening of the cationic polymer coat.

In one embodiment, the present invention provides a pharmaceutical composition, comprising: a core comprising an active ingredient or pharmaceutically acceptable salt thereof; a cationic polymer containing coating over the core and an additional coating over the cationic polymer containing coating, the additional coating comprising an acidifying agent capable of causing or accelerating dissolution of the inner cationic polymer layer. Preferably, the pharmaceutical composition is a humidity-resistant pharmaceutical composition. The active ingredient is optionally and preferably (additionally or alternatively) a humidity-sensitive active ingredient. In another embodiment, the cationic polymer containing coating hermetically seals the core. In another embodiment, another agent (other than an acidifying agent) capable of causing or accelerating dissolution of the inner cationic polymer layer is utilized in the additional coating. Each possibility represents a separate embodiment of the present invention.

“Humidity-sensitive active pharmaceutical ingredient” refers, in one embodiment, to an active ingredient subject to humidity-mediated chemical degradation. In another embodiment, the term includes an active ingredient subject to change into a different polymorph or crystal form as a result of exposure to humidity. Each possibility represents a separate embodiment of the present invention.

“Humidity-sensitive core” refers, in another embodiment, to a pharmaceutical core formulation subject to humidity-mediated chemical or physical modification.

“Humidity-sensitive composition” refers, in yet another embodiment, to a pharmaceutical composition subject to humidity-mediated chemical or physical modification.

In another embodiment, the active ingredient according to the present invention is intended to be immediately released in the stomach of a subject, as opposed to the duodenum, colon, or any other location in the gastrointestinal tract (e.g. in order to exhibit an optimal therapeutic effect). “Optimal therapeutic effect,” in one embodiment, refers to a better or enhanced therapeutic effect, and/or an effect having a more rapid onset, when released in the stomach as opposed to the other locations. In one embodiment, the optimal therapeutic effect is assessed in the average subject. In another embodiment, the optimal therapeutic effect is assessed in the average subject of a particular patient subpopulation targeted by the pharmaceutical composition. Each possibility represents a separate embodiment of the present invention.

In another embodiment, the present invention is suitable for humidity-sensitive formulations wherein the active ingredient is not necessarily humidity-sensitive, but one or more excipients is humidity-sensitive. In another embodiment, both the active ingredient and one or more excipients are humidity-sensitive. “Humidity-sensitive excipient,” as used herein, refers, in one embodiment, to an excipient whose advantageous properties are compromised by exposure to humidity. For instance, the hardness and friability of cores containing high amounts of starch would rapidly decrease if they are not suitably protected from humidity. In another embodiment, the term “humidity-sensitive excipient” includes excipients subject to humidity-mediated chemical degradation. Each possibility represents a separate embodiment of the present invention.

One purpose of the present invention is to provide a formulation that protects the active material and/or the core against humidity by sealing the core, to prevent the humidity penetration into the core. The acidifying agent according to the present invention confers an essentially pH-independent release profile (e.g. immediate release over a wide range of pH, from acidic to neutral conditions). pH-independent release is important, inter alia, in the case wherein the dosage form is expelled from the stomach before it disintegrates, or the pH in the stomach is less acidic than usual, due to e.g. the presence of food.

As provided herein, dosage forms of the present invention confer very effective moisture protection without exhibiting a modified dissolution profile (same or at least highly similar dissolution profile and same or at least highly similar homogeneity) relative to uncoated cores, over a wide range of pH conditions, preferably the entire range of pH conditions between acidic and neutral, and probably into the basic pH range as well. In another embodiment, the dosage forms exhibit inter alia one or more of the following benefits: (a) the possibility of storing the product in bulk quantity, in regular or non-sealed packaging, for a relatively long duration under ambient conditions before packaging; (b) ease in the packaging process without the need for low-moisture conditions; (c) possibility of packaging the final product in less expensive packaging containers that need not meet very high standards of moisture protection (e.g. PVC-PVDC blisters or classic plastic bottles etc, as opposed to Alu or Aclar™ blisters). In another embodiment, the dosage forms exhibit additional advantages, as specified hereinbelow.

The cationic polymer according to the present invention is, in another embodiment, a cationic polyamine. In another embodiment, the cationic polymer comprises a polyallylamine or a salt thereof. In another embodiment, the cationic polymer comprises a polyvinylamine or a salt thereof. In another embodiment, the cationic polymer comprises dicyandiamide. In another embodiment, the cationic polymer is a dicyandiamide-polyalkylenepolyamine condensate. In another embodiment, the cationic polymer is a polyalkylenepolyamine-dicyandiamideammonium condensate. In another embodiment, the cationic polymer is a dicyandiamide-formalin condensate. In another embodiment, the cationic polymer is an addition polymer of epichlorohydrin-dialkylamine. In another embodiment, the cationic polymer is any other cationic polyamine known in the art.

In another embodiment, the cationic polymer is a cationic polyacrylamide. In another embodiment, the cationic polymer is a cationic polyethyleneimine. In another embodiment, the cationic polymer is a cationic gelatin.

In another embodiment, the cationic polymer is a copolymer comprising polyamidine.

In another embodiment, the cationic polymer is a cationic starch or polysaccharide. In another embodiment, the cationic polymer is chitosan. In another embodiment, the cationic polymer is cationic polysaccharide. In another embodiment, the cationic polymer is a cationized starch. In another embodiment, the cationic polymer is cationic guar. In another embodiment, the cationic polymer is cationic hydroxypropyl guar. In another embodiment, the cationic polymer is any other cationic starch or polysaccharide known in the art.

In another embodiment, the cationic polymer is an ammonium chloride-containing polymer. In another embodiment, the cationic polymer is a poly(acryloylethyltrimethylammonium chloride). In another embodiment, the cationic polymer is a poly(acrylamidopropyltrimethylammonium chloride) (polyAPTAC). In another embodiment, the cationic polymer is a poly(methacrylamidopropyltrimethylammonium chloride (polyMAPTAC) or a salt thereof. In another embodiment, the cationic polymer is a copolymer of diallyldimethylammoniumchloride-SO₂. In another embodiment, the cationic polymer is a dry blend of PVA with N-(3-chloro-2-hydroxypropyl)-N,N,N-trimethylammonium chloride. In another embodiment, the cationic polymer is QUAT 188™, available from Dow Chemical, an aqueous solution of 3-chloro-2-hydroxypropyltrimethylammonium chloride, containing varying amounts of water and of NaOH. In another embodiment, the cationic polymer is a polymer of diallyldimethyl ammonium chloride (“DADMAC”). In another embodiment, the cationic polymer is a polymer comprising vinylbenzyltrimethyl ammonium chloride. In another embodiment, the cationic polymer comprises (2-methacryloyloxyethyl)trimethyl-ammonium chloride. In another embodiment, the cationic polymer is a copolymer that comprises diallyldimethylammonium chloride. In another embodiment, the cationic polymer is a copolymer that comprises acryloylethyltrimethylammonium chloride or methacrylamidopropyltrimethylammonium chloride in the form of polymerized units. In another embodiment, the cationic polymer is a copolymer that comprises acryloylethyltrimethylammonium chloride or methacrylamidopropyltrimethylammonium chloride in cleaved form. In another embodiment, the cationic polymer is any other ammonium chloride-containing cationic polymer known in the art.

In another embodiment, the cationic polymer is a cationic polyvinyl alcohol (e.g. a methyl chloride quaternary salt of poly(dimethylamino ethyl acrylate/polyvinyl alcohol graft copolymer or a methyl sulfate quaternary salt of poly(dimethylamino ethyl acrylate)/polyvinyl alcohol graft copolymer). In another embodiment, the polyvinyl alcohol comprises a pendant quaternary ammonium salt. In another embodiment, the cationic polymer is any other cationic polyvinyl alcohol known in the art.

In another embodiment, the cationic polymer is a cationic polyvinylpyrrolidone. In another embodiment, the cationic polymer is a copolymer of polyvinylacetate and polyvinylpyrrolidone. In another embodiment, the cationic polymer is a copolymer of polyvinylalcohol and polyvinylpyrrolidone. In another embodiment, the cationic polymer is any other cationic polyvinylpyrrolidone known in the art.

In another embodiment, the cationic polymer is a polyvinylimidazole. In another embodiment, the cationic polymer is a copolymer of vinylimidazole and polyamidine. In another embodiment, the cationic polymer is a copolymer comprising vinylimidazole.

In another embodiment, the cationic polymer is a polyvinyl-containing compound not falling into one of the above classes. In another embodiment, the cationic polymer comprises a cationic polyvinylformamide. In another embodiment, the cationic polymer comprises a cationic polyvinylacetamide. In another embodiment, the cationic polymer comprises a cationic polyvinylmethylformamide. In another embodiment, the cationic polymer comprises a poly(vinylpyridine) or a salt thereof. In another embodiment, the cationic polymer comprises a cationic polyvinylmethylacetamide. In another embodiment, the cationic polymer is any other polyvinyl-containing compound known in the art.

In another embodiment, the cationic polymer is an epichlorohydrin-containing compound. In another embodiment, the cationic polymer is a poly(dimethylamine-co-epichlorohydrin). In another embodiment, the cationic polymer is a poly(dimethylamine-co-epichlorohydrin-co-ethylendiamine). In another embodiment, the cationic polymer is a poly(amidoamine-epichlorohydrin). In another embodiment, the cationic polymer is any other epichlorohydrin-containing compound known in the art.

In another embodiment, the cationic polymer is an N-vinyl polymer. In another embodiment, the cationic polymer is a copolymer that comprises N-vinylformamide. In another embodiment, the cationic polymer is a copolymer that comprises N-vinylacetamide. In another embodiment, the cationic polymer is a copolymer of comprises N-vinylpyrrolidone and N-methyl-N-vinylformamide. In another embodiment, the cationic polymer comprises N-vinylpyrrolidone. In another embodiment, the cationic polymer comprises N-methyl-N-vinylformamide. In another embodiment, the cationic polymer is a copolymer that comprises N-methyl-N-vinylacetamide. In another embodiment, the cationic polymer is any other N-vinyl polymer known in the art.

In another embodiment, the cationic polymer is a salt of one of the above cationic polymers. In another embodiment, the cationic polymer is a combination of one of the above cationic polymers.

Each cationic polymer represents a separate embodiment of the present invention.

In another embodiment, the cationic polymer is a cationic copolymer.

In another embodiment, the cationic polymer is a methacrylate polymer. In another embodiment, the cationic polymer is a methacrylate-based polymer. In another embodiment, the cationic polymer comprises monomers of an amine methacrylate (e.g. having the following structure):

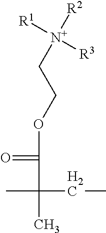

In another embodiment, the cationic polymer comprises monomers of an amine methacrylate subunit. In another embodiment, the cationic polymer comprises monomers of an aminoalkyl methacrylate subunit. In another embodiment, the amine methacrylate is a quaternary ammonium moiety having the general structure below, wherein R¹, R², and R³ are independent selected from alkyl or heteroalkyl moieties:

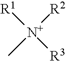

In another embodiment, the amine methacrylate present in the cationic polymer is dimethylaminoethyl methacrylate. In another embodiment, the amine methacrylate polymer is selected from the group consisting of a methyl chloride quaternary salt of a poly(dimethylamino ethyl acrylate/polyvinyl alcohol graft copolymer; a methyl sulfate quaternary salt of a poly(dimethylamino ethyl acrylate)/polyvinyl alcohol graft copolymer; a polymer that comprises dimethylaminoethylmethacrylate; poly(dimethylaminoethylacrylate); a copolymers that comprises dimethylaminoethyl acrylate, and diethylaminoethyl acrylate. In another embodiment, the cationic polymer is a poly(dimethylaminopropylmethacrylamide) (DMAPMAM). In another embodiment, the cationic polymer is a poly(dimethylaminoethylacrylate). In another embodiment, the amine methacrylate is a neutral methacrylic ester available from Rohm Pharma (Degusa) under the name “Eudragit E™.” In another embodiment, the amine methacrylate is any other type of amine methacrylate known in the art. Each possibility represents a separate embodiment of the present invention.

In another embodiment, the cationic polymer according to the present invention comprises monomers of a methacrylic ester. The methacrylic ester according to the present invention is, in another embodiment, a neutral methacrylic ester. In another embodiment, the methacrylic ester is any other type of methacrylic ester known in the art. Each possibility represents a separate embodiment of the present invention.

In another embodiment, the cationic copolymer is a copolymer comprising monomers of an aminoalkyl methacrylate subunit and monomers of a methacrylic ester subunit.

The acidifying agent according to the present invention is, in another embodiment, citric acid. In another embodiment, the acidifying agent is an organic acid. In another embodiment, the acidifying agent is selected from the group consisting of N-acetylglutamic acid, adipic acid, aldaric acid, alpha-ketoglutaric acid, aspartic acid, azelaic acid, camphoric acid, creatine-alpha ketoglutarate, diglycolic acid, dimercaptosuccinic acid, fumaric acid, glutaconic acid, glutamic acid, glutaric acid, isophthalic acid, itaconic acid, maleic acid, malic acid, malonic acid, meglutol, mesaconic acid, mesoxalic acid, 3-methylglutaconic acid, muconic acid, oxalic acid, oxaloacetic acid, pamoic acid, phthalic acids, pimelic acid, sebacic acid, suberic acid, succinic acid, tartaric acid, tartronic acid, terephthalic acid, traumatic acid, methanoic acid, ethanoic acid, propanoic acid, butanoic acid, pentanoic acid, hexanoic acid, heptanoic acid, octanoic acid, nonanoic acid, decanoic acid, dodecanoic acid, hexadecanoic acid, octadecanoic acid, acrylic acid, a fatty acid, docosahexaenoic acid, eicosapentaenoic acid, aspartic acid, glutamic acid, a keto acid, an aromatic carboxylic acid, pyruvic acid, acetoacetic acid, benzoic acid, salicylic acid, dicarboxylic acids, tricarboxylic acids, alpha hydroxy acids, lactic acid, propionic acid, glycolic acid, cinnamic acid, mandelic acid, methanesulfonic acid, ethanesulfonic acid, p-toluenesulfonic acid, and palmatic acid. In another embodiment, the acidifying agent is a combination of one of the above acids. In another embodiment, the acidifying agent is any other acidifying agent known in the art. Each possibility represents a separate embodiment of the present invention.

In another embodiment, the additional coating (i.e. the coating containing the acidifying agent) according to the present invention further comprises a binder. Preferably, the binder is a water-soluble polymer. In another embodiment, the binder is any other type of binder known in the art. Each possibility represents a separate embodiment of the present invention.

In another embodiment, the coating containing the acidifying agent further comprises a glidant. In some preferred embodiments, the glidant is a glidant disclosed herein. In another embodiment, the glidant is any other type of pharmaceutically acceptable glidant known in the art. Each possibility represents a separate embodiment of the present invention.

According to some embodiments one or both of the cationic polymer coating and/or the acidifying agent-containing coating may also optionally contain one or more other pharmaceutically acceptable excipients. Examples of such excipients include but are not limited to plasticizers in either or both coatings, film forming polymers, flavors and sweetening agents.

In another embodiment, a pharmaceutical composition according to the present invention further comprises one or more intermediate coating(s) between the cationic polymer-containing coating and the acidifying agent-containing coating. The number of intermediate coatings is optionally variable as long as the acidifying agent-containing coating can still interact with the cationic polymer coating.

In another embodiment, the intermediate coating comprises a water-soluble polymer. In another embodiment, the intermediate coating comprises a film-forming polymer. In another embodiment, the water-soluble polymer is selected from the group consisting of Povidone (PVP: polyvinyl pyrrolidone), polyvinyl alcohol, a copolymer of PVP and polyvinyl acetate, HPC (hydroxypropyl cellulose) (preferably a low molecular weight HPC), HPMC (hydroxypropyl methylcellulose) (preferably a low molecular weight HPMC), carboxy methyl cellulose (preferably a low molecular weight carboxy methyl cellulose), ethylcellulose, hydroxyethyl cellulose, gelatin, polyethylene oxide, acacia, dextrin, magnesium aluminum silicate, starch, polyacrylic acid, polyhydroxyethylmethacrylate (PHEMA), a gum (e.g. a water-soluble gum), and a polysaccharide. In another embodiment, the water-soluble polymer is a combination of any other above polymers. In another embodiment, the water-soluble polymer is any other pharmaceutically acceptable polymer known in the art that dissolves or disintegrates in acidic to neutral medium. Each possibility represents a separate embodiment of the present invention.

In another embodiment, the intermediate coating is water soluble. Preferably, the intermediate coating generally is soluble and/or able to readily disaggregate in acidic to neutral medium. In another embodiment, the intermediate coating generally is soluble and/or able to readily disaggregate in aqueous medium. Each possibility represents a separate embodiment of the present invention.

In another embodiment, the intermediate coating enhances the stability of the pharmaceutical composition to degradation of the cationic polymer containing coating (e.g. during the further additional acidic agent containing coating process and/or during stability). In another embodiment, the intermediate coating enhances the stability of the pharmaceutical composition to degradation of the active ingredient. In another embodiment, the degradation referred to is mediated or aggravated by high ambient humidity. In another embodiment, the degradation referred to is mediated or aggravated by elevated temperature. In another embodiment, the degradation referred to is a result of the integrity of the cationic polymer containing coating being compromised by the acidifying agent. Each possibility represents a separate embodiment of the present invention.

In another embodiment, a pharmaceutical composition according to the present invention comprises one or many additional coating(s) over the core, applied before the cationic polymer coating, for example optionally a coating to protect the core from the cationic polymer and/or a functional coating, for example a sugar coat or a polymer coat which protects the core from the cationic coating. A functional coating may also optionally be an enteric-coat or a delayed release coat or a modified release coat or any other coat. For instance, if the formula is coated with a modified release coat or an enteric coat which is not enough effective to protect the core from humidity and/or oxidation, optionally a further cationic polymer containing coating (for example containing Eudragit-E) and a further acidic agent containing coating may be applied which will provide excellent humidity and/or oxidation protection but which will immediately dissolve in the body and allow the functional modified release coating or the enteric coating to “function” in vivo as if the tablet had not been further coated with the additional coatings.

In yet another embodiment, a pharmaceutical composition according to the present invention comprises one or many additional coatings, optionally an outer coating, over the additional (acidifying agent-containing) coating. If no intermediate coating is present between the first (cationic polymer-containing) coating and the acidifying agent-containing coating, an additional outer coating will be a third coating; if an intermediate coating is present, an additional outer coating will be a fourth layer. If the inner core was already coated with a first coating before the cationic polymer-containing coating, an additional outer coating will be a fourth or a fifth coating. All such possibilities are generically referred to herein as an “outer coating.” However, this term as used herein does not preclude the presence of an additional coating, applied over the coating described herein as an “outer coating.” In another embodiment, the outer coating comprises a water-soluble polymer or a polymer that disintegrates in water. In another embodiment, the outer coating comprises a cationic polymer. In another embodiment, the outer coating comprises a taste-masking agent. In another embodiment, the cationic polymer and taste-masking agent are in two separate outer layers, applied over the other layers disclosed herein (i.e. over the cationic polymer containing coating, optional intermediate coating, and acidifying agent-containing coating). In another embodiment, the cationic polymer and taste-masking agent are in a single outer layer. Each possibility represents a separate embodiment of the present invention.

In another embodiment, the outer coating comprises a water-soluble or disintegrating polymer. In another embodiment, the outer coating comprises a film-forming polymer. In another embodiment, the water-soluble or disintegrating polymer is selected from the group consisting of Povidone (PVP: polyvinyl pyrrolidone), polyvinyl alcohol, a copolymer of PVP and polyvinyl acetate, HPC (hydroxypropyl cellulose) (preferably a low molecular weight HPC), HPMC (hydroxypropyl methylcellulose) (preferably a low molecular weight HPMC), carboxy methyl cellulose (preferably a low molecular weight carboxy methyl cellulose), ethylcellulose, hydroxyethyl cellulose, gelatin, polyethylene oxide, acacia, dextrin, magnesium aluminum silicate, starch, polyacrylic acid, polyhydroxyethylmethacrylate (PHEMA), a gum (e.g. a water-soluble gum), and a polysaccharide. In another embodiment, the water-soluble or disintegrating polymer is a combination of any other above polymers. In another embodiment, the water-soluble or disintegrating polymer is any other pharmaceutically acceptable polymer known in the art that dissolves in acidic medium. Each possibility represents a separate embodiment of the present invention.

In another embodiment, the outer coating is water soluble. Preferably, the outer coating generally is soluble and/or able to readily disaggregate in acidic or neutral medium. In another embodiment, the outer coating generally is soluble and/or able to readily disaggregate in aqueous medium. Each possibility represents a separate embodiment of the present invention.

In another embodiment one or more of the above coating layer(s) according to the present invention further comprises a plasticizer. In another embodiment, the plasticizer is a selected from the group consisting of dibutyl sebacate, polyethylene glycol, polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol. In another embodiment, each layer can contain a combination of more than one of the above compounds. Each possibility represents a separate embodiment of the present invention.

Preferably, the pharmaceutical composition according to the present invention is an immediate-release pharmaceutical composition. In another embodiment, the pharmaceutical composition is a delayed-onset or slow release pharmaceutical composition. In another embodiment, the pharmaceutical composition is a gastric resistant composition which exhibits delayed release in gastric fluid. In another embodiment, the pharmaceutical composition is any other type of pharmaceutical composition known in the art. Each possibility represents a separate embodiment of the present invention.

In another embodiment, the pharmaceutical composition according to the present invention is in the form of a tablet. In another embodiment, the pharmaceutical composition is in the form of a capsule. In another embodiment, the pharmaceutical composition is in the form of a caplet. In another embodiment, the pharmaceutical composition is in the form of a pellet. In another embodiment, the pharmaceutical composition is any other type of pharmaceutical dosage form known in the art that is appropriate for compositions of the present invention. Each possibility represents a separate embodiment of the present invention.

### Production Methods

In another embodiment, the present invention provides a method for preparation of a pharmaceutical composition, comprising the steps of: (a) applying a cationic polymer containing coating over a pharmaceutical dosage form core comprising an active ingredient or pharmaceutically acceptable salt thereof; and (b) applying an acidifying agent containing coating over the cationic polymer containing coating. The pharmaceutical composition and/or a portion thereof, such as the core for example, is optionally and preferably humidity-sensitive. The active ingredient is optionally and preferably (additionally or alternatively) a humidity-sensitive active ingredient. In another embodiment, the active ingredient is a statin. In another embodiment, the active ingredient is any other active ingredient known in the art. In another embodiment, the core is coated with additional coating(s) before application of the cationic polymer coating, and/or between the cationic polymer and the acidic coatings and/or above the acidic coating. Each possibility represents a separate embodiment of the present invention.

The cationic polymer according to the present invention is, in another embodiment, a cationic polyamine. In another embodiment, the cationic polymer comprises a polyallylamine or a salt thereof. In another embodiment, the cationic polymer comprises a polyvinylamine or a salt thereof. In another embodiment, the cationic polymer comprises dicyandiamide. In another embodiment, the cationic polymer is a dicyandiamide-polyalkylenepolyamine condensate. In another embodiment, the cationic polymer is a polyalkylenepolyamine-dicyandiamideammonium condensate. In another embodiment, the cationic polymer is a dicyandiamide-formalin condensate. In another embodiment, the cationic polymer is an addition polymer of epichlorohydrin-dialkylamine. In another embodiment, the cationic polymer is any other cationic polyamine known in the art.

In another embodiment, the cationic polymer is a cationic polyacrylamide. In another embodiment, the cationic polymer is a cationic polyethyleneimine. In another embodiment, the cationic polymer is a cationic gelatin.

In another embodiment, the cationic polymer is a cationic polyvinyl alcohol (e.g. a methyl chloride quaternary salt of poly(dimethylamino ethyl acrylate/polyvinyl alcohol graft copolymer or a methyl sulfate quaternary salt of poly(dimethylamino ethyl acrylate)/polyvinyl alcohol graft copolymer). In another embodiment, the polyvinyl alcohol comprises a pendant quaternary ammonium salt. In another embodiment, the cationic polymer is any other cationic polyvinyl alcohol known in the art.

In another embodiment, the cationic polymer is a cationic polyvinylpyrrolidone. In another embodiment, the cationic polymer is a copolymer of polyvinylacetate and polyvinylpyrrolidone. In another embodiment, the cationic polymer is a copolymer of polyvinylalcohol and polyvinylpyrrolidone. In another embodiment, the cationic polymer is any other cationic polyvinylpyrrolidone known in the art.

In another embodiment, the cationic polymer is a polyvinylimidazole. In another embodiment, the cationic polymer is a copolymer of vinylimidazole and polyamidine. In another embodiment, the cationic polymer is a copolymer comprising vinylimidazole.

In another embodiment, the cationic polymer is a copolymer comprising polyamidine.

In another embodiment, the cationic polymer is a cationic starch or polysaccharide. In another embodiment, the cationic polymer is chitosan. In another embodiment, the cationic polymer is cationic polysaccharide. In another embodiment, the cationic polymer is a cationized starch. In another embodiment, the cationic polymer is cationic guar. In another embodiment, the cationic polymer is cationic hydroxypropyl guar. In another embodiment, the cationic polymer is any other cationic starch or polysaccharide known in the art.

In another embodiment, the cationic polymer is an ammonium chloride-containing polymer. In another embodiment, the cationic polymer is a poly(acryloylethyltrimethylammonium chloride). In another embodiment, the cationic polymer is a poly(acrylamidopropyltrimethylammonium chloride) (polyAPTAC). In another embodiment, the cationic polymer is a poly(methacrylamidopropyltrimethylammonium chloride (polyMAPTAC) or a salt thereof. In another embodiment, the cationic polymer is a copolymer of diallyldimethylammoniumchloride-SO₂. In another embodiment, the cationic polymer is a dry blend of PVA with N-(3-chloro-2-hydroxypropyl)-N,N,N-trimethylammonium chloride. In another embodiment, the cationic polymer is QUAT 188™, available from Dow Chemical, an aqueous solution of 3-chloro-2-hydroxypropyltrimethylammonium chloride, containing varying amounts of water and of NaOH. In another embodiment, the cationic polymer is a polymer of diallyldimethyl ammonium chloride (“DADMAC”). In another embodiment, the cationic polymer is a polymer comprising vinylbenzyltrimethyl ammonium chloride. In another embodiment, the cationic polymer comprises (2-methacryloyloxyethyl)trimethyl-ammonium chloride. In another embodiment, the cationic polymer is a copolymer that comprises diallyldimethylammonium chloride. In another embodiment, the cationic polymer is a copolymer that comprises acryloylethyltrimethylammonium chloride or methacrylamidopropyltrimethylammonium chloride in the form of polymerized units. In another embodiment, the cationic polymer is a copolymer that comprises acryloylethyltrimethylammonium chloride or methacrylamidopropyltrimethylammonium chloride in cleaved form. In another embodiment, the cationic polymer is any other ammonium chloride-containing cationic polymer known in the art.

In another embodiment, the cationic polymer is a polyvinyl-containing compound. In another embodiment, the cationic polymer comprises a cationic polyvinylformamide. In another embodiment, the cationic polymer comprises a cationic polyvinylacetamide. In another embodiment, the cationic polymer comprises a cationic polyvinylmethylformamide. In another embodiment, the cationic polymer comprises a poly(vinylpyridine) or a salt thereof. In another embodiment, the cationic polymer comprises a cationic polyvinylmethylacetamide. In another embodiment, the cationic polymer is any other polyvinyl-containing compound known in the art.

In another embodiment, the cationic polymer is an epichlorohydrin-containing compound. In another embodiment, the cationic polymer is a poly(dimethylamine-co-epichlorohydrin). In another embodiment, the cationic polymer is a poly(dimethylamine-co-epichlorohydrin-co-ethylendiamine). In another embodiment, the cationic polymer is a poly(amidoamine-epichlorohydrin). In another embodiment, the cationic polymer is any other epichlorohydrin-containing compound known in the art.

In another embodiment, the cationic polymer is an N-vinyl polymer. In another embodiment, the cationic polymer is a copolymer that comprises N-vinylformamide. In another embodiment, the cationic polymer is a copolymer that comprises N-vinylacetamide. In another embodiment, the cationic polymer is a copolymer of comprises N-vinylpyrrolidone and N-methyl-N-vinylformamide. In another embodiment, the cationic polymer comprises N-vinylpyrrolidone. In another embodiment, the cationic polymer comprises N-methyl-N-vinylformamide. In another embodiment, the cationic polymer is a copolymer that comprises N-methyl-N-vinylacetamide. In another embodiment, the cationic polymer is any other N-vinyl polymer known in the art.

In another embodiment, the cationic polymer is a salt of one of the above cationic polymers. In another embodiment, the cationic polymer is a combination of one of the above cationic polymers.

Each cationic polymer represents a separate embodiment of the present invention.

In another embodiment, the cationic polymer of the above method is a methacrylate polymer. In another embodiment, the cationic polymer is a methacrylate-based polymer. In another embodiment, the cationic polymer comprises monomers of an amine methacrylate.

In another embodiment, the cationic polymer of the above method comprises monomers of an amine methacrylate subunit. In another embodiment, the amine methacrylate polymer is selected from the group consisting of a methyl chloride quaternary salt of a poly(dimethylamino ethyl acrylate/polyvinyl alcohol graft copolymer; a methyl sulfate quaternary salt of a poly(dimethylamino ethyl acrylate)/polyvinyl alcohol graft copolymer; a polymer that comprises dimethylaminoethylmethacrylate; poly(dimethylaminoethylacrylate); a copolymers that comprises dimethylaminoethyl acrylate, and diethylaminoethyl acrylate. In another embodiment, the cationic polymer comprises monomers of an aminoalkyl methacrylate subunit. In another embodiment, the amine methacrylate is a quaternary ammonium moiety having the general structure below, wherein R¹, R², and R³ are independently selected from alkyl or heteroalkyl moieties:

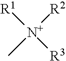

In another embodiment, the amine methacrylate present in the cationic polymer is dimethylaminoethyl methacrylate. In another embodiment, the amine methacrylate is any other amine methacrylate known in the art. Each possibility represents a separate embodiment of the present invention.

In another embodiment, the cationic polymer of the above method comprises monomers of a methacrylic ester. The methacrylic ester according to the present invention is, in another embodiment, a neutral methacrylic ester. In another embodiment, the methacrylic ester is any other type of methacrylic ester known in the art. Each possibility represents a separate embodiment of the present invention.

In another embodiment, the cationic copolymer of the above method is a copolymer comprising monomers of an aminoalkyl methacrylate subunit and monomers of a methacrylic ester subunit. An example of such a cationic copolymer is Eudragit E™.

The acidifying agent according to the present invention is, in another embodiment, citric acid. In another embodiment, the acidifying agent is an organic acid. In another embodiment, the acidifying agent is selected from the group consisting of N-acetylglutamic acid, adipic acid, aldaric acid, alpha-ketoglutaric acid, aspartic acid, azelaic acid, camphoric acid, creatine-alpha ketoglutarate, diglycolic acid, dimercaptosuccinic acid, fumaric acid, glutaconic acid, glutamic acid, glutaric acid, isophthalic acid, itaconic acid, maleic acid, malic acid, malonic acid, meglutol, mesaconic acid, mesoxalic acid, 3-methylglutaconic acid, muconic acid, oxalic acid, oxaloacetic acid, pamoic acid, phthalic acids, pimelic acid, sebacic acid, suberic acid, succinic acid, tartaric acid, tartronic acid, terephthalic acid, traumatic acid, methanoic acid, ethanoic acid, propanoic acid, butanoic acid, pentanoic acid, hexanoic acid, heptanoic acid, octanoic acid, nonanoic acid, decanoic acid, dodecanoic acid, hexadecanoic acid, octadecanoic acid, acrylic acid, a fatty acid, docosahexaenoic acid, eicosapentaenoic acid, aspartic acid, glutamic acid, a keto acid, an aromatic carboxylic acid, pyruvic acid, acetoacetic acid, benzoic acid, salicylic acid, dicarboxylic acids, tricarboxylic acids, alpha hydroxy acids, lactic acid, propionic acid, glycolic acid, cinnamic acid, mandelic acid, methanesulfonic acid, ethanesulfonic acid, p-toluenesulfonic acid, and palmatic acid. In another embodiment, the acidifying agent is a combination of one of the above acids. In another embodiment, the acidifying agent is any other acidifying agent known in the art. Each possibility represents a separate embodiment of the present invention.

In another embodiment, the additional acidifying agent containing coating of the above method further comprises a binder. Preferably, the binder is a water-soluble polymer. In another embodiment, the water soluble polymer that is used as a binder is selected from the group consisting of Povidone (PVP: polyvinyl pyrrolidone), polyvinyl alcohol, a copolymer of PVP and polyvinyl acetate, HPC (hydroxypropyl cellulose) (preferably, a low MW hydroxypropyl cellulose), HPMC (hydroxypropyl methylcellulose) (preferably, of low MW), carboxy methyl cellulose (preferably, of low MW), ethylcellulose, hydroxyethyl cellulose, gelatin, polyethylene oxide, acacia, dextrin, magnesium aluminum silicate, starch, polyacrylic acid, polyhydroxyethylmethacrylate (PHEMA), a gum (e.g. a water-soluble gum), and a polysaccharide. In another embodiment, the binder is any other pharmaceutically acceptable polymer that dissolves in acidic medium. In another embodiment, the binder is a combination of one of the above compounds. In another embodiment, the binder is any other type of binder known in the art. Each possibility represents a separate embodiment of the present invention.

According to some embodiments one or both of the cationic polymer coating and/or the acidifying agent-containing coating may also optionally contain one or more other pharmaceutically acceptable excipients. Examples of such excipients include but are not limited to a plasticizer in either or both coatings and/or film forming polymers and/or flavors and/or sweetening agents.

In another embodiment one or more of the above coating layer(s) according to the present invention further comprises a plasticizer. In another embodiment, the plasticizer is a selected from the group consisting of dibutyl sebacate, polyethylene glycol, polypropylene glycol, dibutyl phthalate, diethyl phthalate, triethyl citrate, tributyl citrate, acetylated monoglyceride, acetyl tributyl citrate, triacetin, dimethyl phthalate, benzyl benzoate, butyl and/or glycol esters of fatty acids, refined mineral oils, oleic acid, castor oil, corn oil, camphor, glycerol and sorbitol. In another embodiment, each layer can contain a combination of more than one of the above compounds. Each possibility represents a separate embodiment of the present invention.

In another embodiment, the above method further comprises the step of applying one or more additional coating(s) over the core, applied before the cationic polymer coating, for example optionally to protect the core from the cationic polymer and/or a functional coating. A protecting coat can be for example a sugar coat or a polymer coat, which protects the core from the cationic coating. A functional coating may also optionally be an enteric-coat or a delayed release coat or a modified release coat or any other coat. For instance, if the formula is coated with a modified release coat or an enteric coat which is not enough effective to protect the core from humidity and or oxidation, optionally a further cationic polymer containing coating (for example containing Eudragit-E) and a further acidic agent containing coat may be applied, which will provide excellent humidity-protection but which will immediately dissolve in the body and allow the functional modified release coating or the enteric coating to “function” in vivo as if the tablet had not been coated with the additional coatings.

In another embodiment, the above method further comprises the step of applying one or more intermediate coatings between the cationic polymer containing coating and the additional acidifying agent containing coating. The number of intermediate coatings is optionally variable as long as the acidifying agent of the acidifying agent-containing coating interacts with the cationic polymer containing coating to speed up and/or improve the homogeneity of its in vivo and/or in vitro dissolution.

In another embodiment, the one or more intermediate coatings enhance the stability of the cationic polymer containing coat during the further coating process and/or the stability under storage of the pharmaceutical composition. In another embodiment, the degradation referred to is mediated or aggravated by high ambient humidity. In another embodiment, the degradation referred to is mediated or aggravated by elevated temperature. In another embodiment, the degradation referred to is a result of the integrity of the cationic polymer containing coating being compromised by the acidifying agent. In another embodiment, the degradation referred to is a spontaneous time-mediated degradative process. Each possibility represents a separate embodiment of the present invention.

In another embodiment, the above method further comprises the step of applying one or more outer coatings over the additional acidifying agent containing coating. Preferably, the outer coating is water soluble and/or readily disaggregates in aqueous solution. In another embodiment, the outer coating comprises a water-soluble polymer. In another embodiment, the outer coating comprises a cationic polymer. In another embodiment, the outer coating comprises a taste-masking agent. Each possibility (and any other possibility not cited here) represents a separate embodiment of the present invention.

Preferably, the above method according to the present invention results in production of an immediate-release pharmaceutical composition. In another embodiment, the above method according to the present invention results in production of a delayed-onset or slow release pharmaceutical composition. In another embodiment, the above method according to the present invention results in production of a gastric resistant composition which exhibits delayed release in gastric fluid. In another embodiment, the above method according to the present invention results in production of any other type of pharmaceutical composition known in the art. Each possibility represents a separate embodiment of the present invention.

In another embodiment, the above method according to the present invention results in production of a tablet. In another embodiment, the above method according to the present invention results in production of a capsule. In another embodiment, the above method according to the present invention results in production of a caplet. In another embodiment, the above method according to the present invention results in production of a pellet. In another embodiment, the above method according to the present invention results in production of any other type of pharmaceutical dosage form known in the art that is appropriate for compositions of the present invention. Each possibility represents a separate embodiment of the present invention.

In another embodiment, the final step or steps of a production method of the present invention (i.e. the step(s) following coating) are performed under ambient humidity and/or temperature conditions. As provided herein, dosage forms of the present invention exhibit superior humidity and environmental resistance, enabling the process to take place even for a long time under ambient and even bad humidity and/or temperature conditions.

In another embodiment, a production method of the present invention further comprises the step of packaging the dosage form. In another embodiment, the packaging step utilizes a packaging material that is not required to meet high standards for humidity resistance. As provided herein, dosage forms of the present invention exhibit superior humidity and environmental resistance, obviating the need for packaging that meets high standards for humidity and/or environmental resistance.

### Active Ingredients

The active ingredient according to the present invention is, in another embodiment, a humidity-sensitive active ingredient. Alternatively, in at least some embodiments, the active ingredient is not itself humidity sensitive but the pharmaceutical composition, or a least a portion thereof (such as the core for example) is humidity sensitive. Alternatively or additionally, the present invention in at least some embodiments protects the core and/or its active agent from oxidation, in which case the acidic agent containing coating increases the in vivo dissolution/opening of the cationic polymer coat.

In another embodiment, the active ingredient is intended to be released in the stomach of a subject. In another embodiment, the active ingredient is a humidity-sensitive active ingredient that is intended to be released in the stomach of a subject. In another embodiment, the active ingredient is intended to be released in another portion of the gastrointestinal tract, apart from the stomach.

In another embodiment, the humidity-sensitive active ingredient is selected from, inter alia, olanzapine, acetysalicylic acid, aminophylline, ascorbic acid, atenolol, betahistine mesylate, calcium chloride, captopril, carbachol, carpronium chloride, cefaclor, cefadroxil, cephrabine, chlorophyllin sodium-copper salt, choline salicylate, choline theophyllinate, citicoline, clindamycin HCl, cyanocobalamin, desipramine HCl, dexamethazone phosphate disodium salt, diclofenac sodium, dimethylaminoethyl ester dihydrochloride, disopyramide phosphate, divalproex sodium, ethionamide, fenoprofen calcium, gemfibrozil, hexamethonium bromide, isosorbide, L-proline meprobamate, methocarbamol, methyldopa, oxtriphylline, oxytetracycline HCl, panthenol, piracetam, plant extracts (Querci Folium extract, Mallot Cortex extract, Equisetum arvense extract, etc.), procainamide HCl, procainamide hydrochloride, ranitine HCl, reserpilic acid, rifamprin, lincomycin HCl, sodium valproate, tetracycline HCl, thiamine HCl, clavulenic acid and salts thereof, polymixin, herbals, herbal extracts, nutritional products, nitroglycerin, alkaloid salts, streptomycin, idoxuridine, and tolazoline hydrochloride.

In another embodiment, the humidity-sensitive active ingredient is selected from an anticholestrolemic drug and a proton-pump inhibitor.

In another embodiment, the active ingredient according to the present invention is selected from, inter alia, water soluble and water insoluble drugs. Non-limiting examples of such therapeutically active agents include antihistamines (e.g., dimenhydrinate, diphenhydramine, chlorpheniramine and dexchlorpheniramine maleate), analgesics (e.g., aspirin, codeine, morphine, dihydromorphone, oxycodone, etc.), non-steroidal anti-inflammatory agents (e.g., naproxyn, diclofenac, indomethacin, ibuprofen, sulindac), anti-emetics (e.g., metoclopramide), anti-epileptics (e.g., phenyloin, meprobamate and nitrezepam), vasodilators (e.g., nifedipine, papaverine, diltiazem and nicardirine), anti-tussive agents and expectorants (e.g., codeine phosphate), anti-asthmatics (e.g. theophylline), antacids, anti-spasmodics (e.g. atropine, scopolamine), antidiabetics (e.g., insulin), diuretics (e.g., ethacrynic acid, bendrofluazide), anti-hypotensives (e.g., propranolol, clonidine), antihypertensives (e.g., clonidine, methyldopa), bronchodilators (e.g., albuterol), steroids (e.g., hydrocortisone, triamcinolone, prednisone), antibiotics (e.g., tetracycline), antihemorrhoidals, hypnotics, psychotropics, antidiarrheals, mucolytics, sedatives, decongestants, laxatives, vitamins, stimulants (including appetite suppressants such as phenylpropanolamine).

Each active ingredient represents a separate embodiment of the present invention.

In another embodiment, the active ingredient according to the present invention is a statin. In another embodiment, the active ingredient is a pharmaceutically acceptable salt of a statin. In another embodiment, the statin is present in the pharmaceutical composition in a humidity-sensitive form. In another embodiment, the statin is present in the core in a humidity-sensitive form or in a humidity sensitive core. Each possibility represents a separate embodiment of the present invention.

In another embodiment, the statin is atorvastatin. In another embodiment, the atorvastatin is humidity-sensitive form of atorvastatin. In another embodiment, the active ingredient is a pharmaceutically acceptable salt of atorvastatin. In another embodiment, the salt is a salt of an alkaline earth metal. In another embodiment, the alkaline earth metal is calcium or magnesium. In another embodiment, the pharmaceutically acceptable salt is atorvastatin calcium. In another embodiment, the pharmaceutically acceptable salt is any other pharmaceutically acceptable salt of atorvastatin known in the art. Each possibility represents a separate embodiment of the present invention.

In another embodiment, a crystalline form of atorvastatin or a salt thereof is utilized. In another embodiment, the form is crystalline atorvastatin calcium form VI. In another embodiment, amorphous atorvastatin is utilized. In another embodiment, any other salt, crystalline form, or amorphous form of atorvastatin known in the art is utilized. Each possibility represents a separate embodiment of the present invention.

In another embodiment, the statin according to the present invention is any other statin known in the art. Each possibility represents a separate embodiment of the present invention.

In another embodiment, in the case of an atorvastatin-containing composition, the atorvastatin is present in an amount of from about 1% to about 50% weight per weight (w/w) according to the weight of the base. In another embodiment, the atorvastatin is present in an amount of from about 1% to about 30% w/w according to the weight of the base. In another embodiment, the atorvastatin is present in an amount of from about 1% to about 20% w/w according to the weight of the base. In another embodiment, the atorvastatin is present in an amount of from about 1% to about 10% w/w according to the weight of the base. Each possibility represents a separate embodiment of the present invention.

### Excipients

In another embodiment, a dosage form according to the present invention comprises one or more humidity-sensitive excipients.

Unless otherwise indicated, all percentages of ingredients in formulations are expressed as weight by weight (w/w) percent. Also unless otherwise indicated, all percentages of ingredients are expresses as w/w percentage, separately for the core and for the coating (e.g. an ingredient in the core is expressed as w/w percentage for the core alone).

In another embodiment, the core of pharmaceutical compositions according to the present invention comprises a major excipient. In another embodiment, the major excipient is selected from the group consisting of starch, pregelatinized starch, lactose or a combination thereof. In another embodiment, the major excipient is any other excipient known in the art. In another embodiment, the major excipient is present in an amount of at least about 30% of the core. In another embodiment, the major excipient is present in an amount of at least about 50% of the core. In another embodiment, the major excipient is present in an amount of at least about 70% of the core. In another embodiment, the major excipient is present in an amount of at least about 90% of the core. In another embodiment, the core consists essentially of pure active ingredient. Each possibility represents a separate embodiment of the present invention.

In another embodiment, the core of pharmaceutical compositions of the present invention further comprises a major excipient specified hereinabove. In another embodiment, one of the coatings comprises any other excipient known in the art.

In another embodiment, in the case of atorvastatin-containing compositions, the amount of the major excipient is determined according to the form of the atorvastatin (e.g. the particular salt, crystalline form, or amorphous form).

In another embodiment, the core of pharmaceutical compositions of methods and compositions further comprises a minor excipient. In another embodiment, the minor excipient is selected from the group consisting of one or more of HPC, HPMC, PVP, crospovidone, Tween™, magnesium stearate, silicon dioxide, microcrystalline cellulose or Aerosil™. In another embodiment, the minor excipient is any other excipient known in the art. In another embodiment, the minor excipient is present in an amount of up to about 35% of the core. In another embodiment, the minor excipient is present in an amount of up to about 20% of the core. In another embodiment, the minor excipient is present in an amount of up to about 10% of the core. Each possibility represents a separate embodiment of the present invention.

In another embodiment, one of the coatings of pharmaceutical compositions of the present invention further comprises a major excipient specified hereinabove. In another embodiment, one of the coatings comprises any other excipient known in the art.

In another embodiment, the minor excipients according to the present invention are selected from the group consisting of fillers, tableting aids, flow-regulating agents, hardness enhancers, glidants, lubricants, absorption enhancers, binders, and disintegrants.

Examples of suitable binders include but are not limited to Povidone (PVP: polyvinyl pyrrolidone), low molecular weight HPC (hydroxypropyl cellulose), low molecular weight HPMC (hydroxypropyl methylcellulose), low molecular weight carboxymethyl cellulose, ethylcellulose, hydroxyethylcellulose, gelatin, polyethylene oxide, acacia, dextrin, magnesium aluminum silicate, starch, and polymethacrylates. In another embodiment, the binder is selected from HPC and Povidone.

Examples of suitable disintegrants include but are not limited to Crospovidone (cross-linked PVP), pregelatinized starch (e.g. starch 1500), microcrystalline starch, water insoluble starch, calcium carboxymethyl cellulose, magnesium aluminum silicate (Veegum), and combinations thereof. In another embodiment, the disintegrant is pregelatinized starch.

Examples of suitable fillers include but are not limited to microcrystalline cellulose (e.g., Avicel®), starch, lactitol, lactose, dibasic calcium phosphate or any other type of suitable inorganic calcium salt and sucrose, and combinations thereof. In another embodiment, the filler is lactose monohydrate.

Examples of suitable lubricants include but are not limited to stearate salts such as magnesium stearate, calcium stearate, and sodium stearate; stearic acid, talc, sodium stearyl fumarate, and compritol (glycerol behenate), corola oil, glyceryl palmitostearate, hydrogenated vegetable oil, magnesium oxide, mineral oil, poloxamer, polyethylene glycol, polyvinyl alcohol, sodium benzoate, talc, sodium stearyl fumarate, compritol (glycerol behenate) and sodium lauryl sulfate (SLS), and combinations thereof. In another embodiment, the lubricant is magnesium stearate.

Examples of suitable flow-regulating agents include but are not limited to, colloidal silicon dioxide, and aluminum silicate. In another embodiment, the flow-regulating agent is colloidal silicon dioxide.

Examples of suitable hardness enhancers include but are not limited to silicon dioxide, which is known to improve the hardness of pregelatinized starch-containing tablets.

In another embodiment, the core comprises a buffering agent such as, for example, an inorganic salt compound or an organic alkaline salt compound. In another embodiment, the buffering agent is selected from the group consisting of potassium bicarbonate, potassium citrate, potassium hydroxide, sodium bicarbonate, sodium citrate, sodium hydroxide, calcium carbonate, dibasic sodium phosphate, monosodium glutamate, tribasic calcium phosphate, monoethanolamine, diethanolamine, triethanolamine, citric acid monohydrate, lactic acid, propionic acid, tartaric acid, fumaric acid, malic acid, and monobasic sodium phosphate.

The core can also optionally contain at least one of a wetting agent, suspending agent, surfactant, and dispersing agent, or a combination thereof.

Examples of suitable wetting agents include, but are not limited to, poloxamer, polyoxyethylene ethers, polyoxyethylene sorbitan fatty acid esters (polysorbates), polyoxymethylene stearate, sodium lauryl sulfate, sorbitan fatty acid esters, benzalkonium chloride, polyethoxylated castor oil, and docusate sodium.

Examples of suitable suspending agents include but are not limited to alginic acid, bentonite, carbomer, carboxymethylcellulose, carboxymethylcellulose calcium, hydroxyethylcellulose, hydroxypropyl cellulose, microcrystalline cellulose, colloidal silicon dioxide, dextrin, gelatin, guar gum, xanthan gum, kaolin, magnesium aluminum silicate, maltitol, medium chain triglycerides, methylcellulose, polyoxyethylene sorbitan fatty acid esters (polysorbates), polyvinyl pyrrolidone (PVP), propylene glycol alginate, sodium alginate, sorbitan fatty acid esters, and tragacanth.

Examples of suitable surfactants include but are not limited to anionic surfactants such as docusate sodium and sodium lauryl sulfate; cationic surfactants, such as cetrimide; and nonionic surfactants, such as polyoxyethylene sorbitan fatty acid esters (polysorbates) and sorbitan fatty acid esters.

Examples of suitable dispersing agents include but are not limited to poloxamer, polyoxyethylene sorbitan fatty acid esters (polysorbates), and sorbitan fatty acid esters.

The content of the wetting agent, surfactant, dispersing agent and suspending agent can range from about 0 to about 30% of the weight of the formulation, although preferably they are present in an amount of from about 0 to about 10%.

In another embodiment, a pharmaceutical composition according to the present invention further comprises a gel-forming agent. In another embodiment, the gel-forming agent is selected from the group consisting of a cellulose derivative, a vinyl polymer, an acrylic polymer or copolymer, a gum, a protein, a polysaccharide, a polyaminoacid, a polyalcohol, and a polyglycol. In another embodiment, the protein is selected from the group consisting of gelatin and collagen. In another embodiment, the polysaccharide is selected from the group consisting of pectin, pectic acid, alginic acid, sodium alginate, polyaminoacids, polyalcohols, and polyglycols. Each possibility represents a separate embodiment of the present invention.

The tableting agent according to the present invention is, if present, preferably present in an amount of up to about 2%. In another embodiment, the tableting agent is Aerosil™. In another embodiment, the tableting agent is any other tableting agent known in the art. Each possibility represents a separate embodiment of the present invention.

The glidant according to the present invention is, if present, preferably present in an amount of up to about 2%. In another embodiment, the glidant is talc. In another embodiment, the glidant is other glidant known in the art. Each possibility represents a separate embodiment of the present invention.

The surfactant according to the present invention is, if present, preferably present in an amount of up to about 2%. In another embodiment, the surfactant is Tween®. In another embodiment, the surfactant is other surfactant known in the art. Each possibility represents a separate embodiment of the present invention.

The lubricant according to the present invention is, if present, preferably present in an amount of up to about 2%. In another embodiment, the lubricant is magnesium stearate. In another embodiment, the lubricant is other lubricant known in the art. Each possibility represents a separate embodiment of the present invention.

### Stabilizers

In another embodiment, preferably for pharmaceutical compositions containing atorvastatin and more preferably a calcium salt thereof, the pharmaceutical composition according to the present invention are free of significant amounts of a stabilizer such as CaCO₃ or CaCl₂. In another embodiment, the stabilizer is present in an amount of up to about 10%. In another embodiment, the stabilizer is present in an amount of up to about 5%. Each possibility represents a separate embodiment of the present invention.

In another embodiment, “stabilizer” refers to a compound selected from the group consisting of basifying agents and buffering agents.

### Optionally Absent Excipients

In another embodiment, preferably for pharmaceutical compositions containing atorvastatin and more preferably a calcium salt thereof, excipients such as croscarmellose sodium, carmellose calcium, sodium starch glycolate and stearic acid are absent or, if present, are present in sufficiently low quantities so as to be unable to influence stability of the active ingredient. With regard to these excipients, the amount depends upon such factors as whether they are combined with the active ingredient during a wet or dry process while producing the core, and also with regard to the temperature to which the formulation is exposed during this processing. If a wet process is used, such as wet granulation for example, preferably these excipients are not used at least during the wet portion of such processing, or if used, are preferably present in an amount of only up to about 10% or less.

### Therapeutic Methods

In another embodiment, the present invention provides a method for lowering the cholesterol level of a subject in need thereof, comprising the step of administering to the subject a pharmaceutical composition of the present invention, thereby lowering the cholesterol level of a subject.

In another embodiment, the present invention provides a pharmaceutical composition for lowering the cholesterol level of a subject, wherein the pharmaceutical composition is as described hereinabove.

### Additional Advantageous Properties

In another embodiment, atorvastatin-containing formulations of the present invention (or compositions containing an atorvastatin salt or atorvastatin free base in amorphous or any known crystal form) remain stable to environmental influences even in the absence of significant amounts of stabilizers, such as alkalizing agents, buffering agents, etc. An example of a stabilizer is CaCO₃.

As provided herein in the Examples section, pharmaceutical compositions according to the present invention exhibit, in another embodiment, improved resistance to humidity, relative to compositions lacking the combination of a cationic polymer overcoated with a layer containing an acidifying agent, as exemplified herein. In another embodiment, the coating of the pharmaceutical composition is able to confer protection of a humidity-sensitive active ingredient (e.g. atorvastatin calcium) such that fewer than 0.25% atorvastatin lactone is generated after incubation at 40° C./75% RH for six month. In another embodiment, fewer than 0.35% total impurities are generated after incubation at 40° C./75% RH for six month. Each possibility represents a separate embodiment of the present invention.

In another embodiment, provided herein in the Examples section, pharmaceutical compositions according to the present invention exhibit improved shelf life, relative to compositions lacking the combination of a cationic polymer overcoated with a layer containing an acidifying agent, as exemplified herein. In another embodiment, the pharmaceutical compositions protect a humidity-sensitive active ingredient from significant degradation after incubation at 40° C./75% RH for six month. Such improvements would particularly be provided when the pharmaceutical composition is packaged in regular packaging material which poorly protects the humidity sensitive active ingredient and/or composition from humidity. Each possibility represents a separate embodiment of the present invention.

In another embodiment, provided herein in the Examples section, pharmaceutical compositions according to the present invention exhibit an improved in vitro release profile in mildly acidic or neutral (and probably alkaline) solutions, relative compositions coated only with the cationic polymer, as exemplified herein. In another embodiment, the average time of release is similar to uncoated pharmaceutical compositions. In another embodiment, the relative standard deviation of the release is low. In another embodiment, the relative standard deviation of the release of an immediate release composition of the invention is less than about 2% in an in vitro dissolution test after a 45-minute, preferably after 30 minute, more preferably after 20 minute and most preferably after 10 minute incubation under physiological conditions as are known in the art for standardized dissolution testing. In another embodiment, the relative standard deviation of the release is less than about 2% after a 45-minute, preferably after 30 minute, more preferably after 20 minute and most preferably after 10 minute incubation under gastric juice-like conditions. Each possibility represents a separate embodiment of the present invention.

In another embodiment, provided herein in the Examples section, pharmaceutical compositions according to the present invention exhibit lower water uptake under humid conditions, relative to compositions lacking the combination of a cationic polymer overcoated with a layer containing an acidifying agent, as exemplified herein.

### Cationic Polymer-Containing Coatings

A non-limiting example of a cationic polymer-containing coating suitable for methods and compositions of the present invention is Eudragit E™. Eudragit E™ is a copolymer of dimethylaminoethyl methacrylate and neutral methacrylic esters, and is available from Rohm Pharma (Degusa).

### Optional Further Coatings

A non-limiting example of a further coating suitable for methods and compositions of the present invention is Opadry II™. Opadry II™ is a dry-blend system comprising a polymer and a plasticizer that adds flexibility to the coating. Specifically, Opadry II™ comprises HPMC (polymer), Lactose (film-enhancing excipient), Polyethylene Glycol (plasticizer), pigments, and optionally polydextrose (film enhancing excipient and co-polymer). Without limitation, the further coating may optionally be a subcoating, intermediate coating and/or outer coating as described herein.

In another embodiment, the further coating comprises another polymer, e.g. polyvinyl alcohol, a water-soluble synthetic polymer having the formula (C₂H₄O)ₙ and the structure:

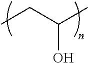

In another embodiment, one or more further coats can also be functional coats such as for instance enteric coating polymers, delayed or modified release coats below the cationic polymer containing coat and so forth.

### General Coating Properties (Optional)

In another embodiment, any of the above described coatings according to the present invention can comprise a plasticizer. In another embodiment, the plasticizer adds flexibility to the coating. In another embodiment, the increased flexibility enables the coating to withstand cycles of expansion and contraction of the core, due to temperature fluctuation, etc. Each possibility represents a separate embodiment of the present invention.

An additional advantage of pharmaceutical compositions of the present invention is their ability to contain an increased amount of cationic polymer-containing coating, thus providing improved moisture protection without compromising speed and homogeneity of drug release. As provided herein in the examples, addition of 5 mg Eudragit E to atorvastatin-containing cores conferred partial moisture protection. By increasing the amount of Eudragit E coat, the tablets would have exhibited better moisture protection, but (without the addition of the acid agent containing coat) the dissolution profile of the tablets, particularly in slightly acidic and neutral medium, would have been significantly prolonged, as provided herein in the examples.

Thus, in another embodiment, the cationic polymer-containing coating of dosage forms of the present invention is present in an amount up to 20 mg or less, preferably up to 30 mg or less, and more preferably up to 50 mg or less (for a regular 300 to 350 mg inner core tablet). In another embodiment, the cationic polymer-containing coating is present in an amount of about 30 mg or less for a 12 mm*6 mm oblong 350 mg core. It is well within the ability of those skilled in the art to determine the corresponding amount of coating for a core of different size. Since surface area generally varies with the square of the radius (e.g. the surface area of a sphere is [4×π×r²]), and volume generally varies with the cube of the radius, the amount of corresponding amount of coating can be readily calculated by those skilled in the art.

The following experimental details are intended to provide non-limiting exemplification of the principles of the invention.

### EXPERIMENTAL DETAILS SECTION

### Example 1

### Production of Humidity-Resistant 10, 20, and 40 mg Atorvastatin Tablets with a Release Profile Similar to Uncoated Tablets

Experiments were conducted to develop immediate-release, humidity-resistant atorvastatin dosage forms (containing atorvastatin base as atorvastatin Ca in the core) with a release profile similar to uncoated tablets. 10, 20, and 40 mg atorvastatin tablets coated with Eudragit E™ (thus humidity-resistant) and also containing a citric acid-containing layer were produced. The tablets had the following composition:

TABLE 1A
Composition of the 10, 20, and 40 mg atorvastatin (as Atorvastatin Ca)
tablets in milligrams (mg).

| | | Dosage | | | % in the | |
|---|---|---|---|---|---|---|
| | | 10 mg | 20 mg | 40 mg | formula | Function |
| Core | Atorvastatin | 87.50 | 175.00 | 350.00 | 87.94% | Inner |
| (mg) | Ca-containing core | | | | | immediate |
| | | | | | | release core |
| Cationic coat | Eudragit E ™ | 3.00 | 6.00 | 12.00 | 3.02% | Protects core |
| (mg) | | | | | | |
| Intermed. | Opadry II | 3.00 | 6.00 | 12.00 | 3.02% | Protects |
| coat. | (white) ™ | | | | | cationic coat |
| (mg) | | | | | | |
| Acid | Citric Acid | 2.25 | 4.50 | 9.00 | 2.26% | Acidifying |
| coat | | | | | | agent |
| (mg) | Povidone 30 | 0.12 | 0.24 | 0.48 | 0.12% | Binder |
| | Aerosil | 0.63 | 1.26 | 2.52 | 0.63% | Glidant |
| Outer coat | Opadry II | 3.00 | 6.00 | 12.00 | 3.02% | Taste |
| (mg) | (white) ™ | | | | | masking |
| | TOTAL (mg) | 99.5 | 199.0 | 398.0 | 100.0% | |

The tablets were produced as follows:

### Core Production

TABLE 1B
Core compositions

| | | | | | % in the | |
|---|---|---|---|---|---|---|
| | | 10 mg | 20 mg | 40 mg | formula | function |
| Granulation blend | Atorvastatin | 10.36 | 20.72 | 41.44 | 10.41% | API |
| | Ca | | | | | |
| | Form P* | | | | | |
| | Starch 1500 | 43.75 | 87.50 | 175.00 | 43.97% | Filler/ |
| | | | | | | disintegrant |
| | Cros- | 24.03 | 48.06 | 96.11 | 24.15% | Super- |
| | povidone | | | | | disintegrant |
| | Lactose | 2.19 | 4.38 | 8.75 | 2.20% | Filler |
| | mono- | | | | | |
| | hydrate | | | | | |
| | 100M | | | | | |
| Granulation | Tween 80 | 0.35 | 0.70 | 1.40 | 0.35% | Surfactant |
| solution | Klucel LF | 1.66 | 3.33 | 6.65 | 1.67% | Binder |
| | or EF | | | | | |
| | Water I | — | — | — | | |
| | Ethanol | — | — | — | | |
| Extra- | Cros- | 2.19 | 4.38 | 8.75 | 2.20% | Super- |
| granular | povidone | | | | | disintegrant |
| excipients | Lactose DC | 2.19 | 4.38 | 8.75 | 2.20% | Filler |
| | Mg stearate | 0.35 | 0.70 | 1.40 | 0.35% | Lubricant |
| | Aerosil | 0.44 | 0.88 | 1.75 | 0.44% | Binder and |
| | | | | | | glidant |
| Cationic | Eudragit E | 3.00 | 6.00 | 12.00 | 3.02% | Protects |
| coat (mg) | | | | | | core |
| Intermed. | Opadry II | 3.00 | 6.00 | 12.00 | 3.02% | Protects |
| coat. | (white) | | | | | cationic coat |
| (mg) | | | | | | |
| Acid coat | Citric acid | 2.25 | 4.50 | 9.00 | 2.26% | Acidifying |
| (mg) | | | | | | agent |
| | Povidone 30 | 0.12 | 0.24 | 0.48 | 0.12% | Binder |
| | Aerosil | 0.63 | 1.26 | 2.52 | 0.63% | Glidant |
| Outer | Opadry II | 3.00 | 6.00 | 12.00 | 3.02% | Taste |
| coat (mg) | (white) | | | | | masking |
| | TOTAL | 99.5 | 199 | 398 | 100% | |

The pH of the formula milled and suspended in 40 ml deionized water is in the range of 3.5-5.3.

Steps in production of core: (a) premix of the granulation blend; (b) granulation of the premix under shear using a granulation solution (water/ethanol 50:50) containing the Tween 80 and Klucel LF; (c) drying of the wet granulate; (d) milling of the dry granulate; (e) loading back and mixing of the granulate with the extragranular excipients (except Mg Stearate); (f) final mix with the Mg stearate; and (g) compression of the tablets.

### Coating

Eudragit E coating solution: 49.5 g of Eudragit E™ was dissolved in 775.5 g ethanol.

Opadry II coating suspension: 99 g of Opadry II (white)™ was mixed vigorously for 1 hour in 630 g purified water USP.

Acid coat coating suspension: 1.98 g Povidone K30 and 37.13 g citric acid anhydrous were dissolved in 21 g purified water USP and 171 g ethanol. After complete dissolution, 10.39 g Aerosil™ was added, and the coating suspension was mixed vigorously until the end of the coating process.

Coating process: for each dosage, 467.5 g of uncoated cores were coated in a small-scale perforated pan coater successively with 1) the Eudragit E coating solution, 2) the Opadry II coating suspension, 3) the acid coat coating suspension and 4) a second coat of the Opadry II coating suspension. The coating process was performed by spraying the coating solution/suspension on the tablet bed during continuous drying by a hot air stream.

### Example 2

### Dissolution Profile of 10, 20, and 40 mg Atorvastatin Tablets Under Gastric Juice-Like Conditions

As shown in Table 2, in vitro dissolution studies of the 10, 20, and 40 mg atorvastatin tablets from Example 1 (“coated”) were performed under gastric juice-like conditions, i.e. in 0.1 N HCl, with a paddle rotating at 75 rpm (6 tablets tested for each lot). The four coatings present on the tablets were not found to greatly delay dissolution of the tablets, conferring only approximately a five-minute lag relative to uncoated cores.

The RSD (relative standard deviation) of the 40 mg dosage form were quite low, even at the early time points (e.g. 10 minutes). Similarly low RSD values were obtained with the lower 10 mg and 20 mg dosage forms.

TABLE 2
Dissolution profiles (as % of API dissolution) of the of 10, 20,
and 40 mg atorvastatin tablets in 0.1N HCl.

| | Dosage | | | | | |
|---|---|---|---|---|---|---|
| | 10 mg | | 20 mg | | | |
| | atorvastatin (as | | atorvastatin (as | | | |
| | Atorvastatin | | Atorvastatin | | 40 mg | |
| | Ca) | | Ca) | | atorvastatin (as | |
| | Un- | | Un- | | Atorvastatin Ca) | |
| Min | coated | Coated | coated | Coated | Uncoated | Coated |
| 0 | 0 | 0 | 0 | 0 | 0 | 0 |
| 5 | 8.9 | 16 | 26.3 | 5 | 14.2 | 5 (RSD 53%) |
| 10 | 35.4 | 45 | 35.4 | 38 | 27.4 | 41 (RSD 2.4%) |
| 15 | 49.4 | 59 | 46.8 | 53 | 35.0 | 52 (RSD 1.4%) |
| 20 | 57.5 | 66 | 53.8 | 61 | 40.3 | 56 (RSD 1.5%) |
| 30 | 65.5 | 74 | 59.3 | 68 | 44.0 | 60 (RSD 0.9%) |
| 45 | 73.3 | 81 | 64.3 | 73 | 46.5 | 61 (RSD 0.7%) |

### Example 3

### Dissolution Profile of 10, 20, and 40 mg Atorvastatin Tablets Under Neutral Conditions

As shown in Table 3, in vitro dissolution studies of the 10, 20, and 40 mg atorvastatin tablets from Example 1 (“coated”) were performed under neutral conditions, similar to conditions in the small intestine, i.e. in IF (phosphate buffer simulating intestinal fluid, pH 6.8), with a paddle rotating at 75 rpm (6 tablets tested for each lot). The four coatings present on the tablets were not found to greatly delay dissolution of the tablets, conferring only approximately a five-minute lag relative to uncoated cores.

The RSD (relative standard deviations) of the 40 mg dosage form were quite low, even at the early time points (e.g. 10 minutes). Similarly low RSD values were obtained with the lower 10 mg and 20 mg dosage forms.

TABLE 3
Dissolution profiles (as % of API dissolution) of the of 10, 20, and 40 mg
atorvastatin tablets at pH 6.8.

| | Dosage | | | | | |
|---|---|---|---|---|---|---|
| | 10 mg | | 20 mg | | | |
| | atorvastatin (as | | atorvastatin (as | | | |
| | Atorvastatin | | Atorvastatin | | 40 mg | |
| | Ca) | | Ca) | | atorvastatin (as | |
| | Un- | | Un- | | Atorvastatin Ca) | |
| Min | coated | Coated | coated | Coated | Uncoated | Coated |
| 0 | NP | 0 | 0 | 0 | 0 | 0 |
| 5 | NP | 58 | 83 | 8 | 87 | 10 (RSD 59%) |
| 10 | NP | 91 | 95 | 78 | 95 | 88 (RSD 1.5%) |
| 15 | NP | 94 | 95 | 90 | 96 | 94 (RSD 1.5%) |
| 20 | NP | 95 | 96 | 93 | 97 | 95 (RSD 1.1%) |
| 30 | NP | 96 | 96 | 94 | 97 | 96 (RSD 0.9%) |
| 45 | NP | 97 | 96 | 95 | 98 | 98 (RSD 0.8%) |

### Example 4

### Tablets of the Present Invention Dissolve More Homogeneously Under Neutral Conditions than Tablets Coated with Eudragit E™ Alone

Dissolution profiles of tablets of the present invention (40 mg Atorvastatin coated tablets coated with 4 layers from example 1) under neutral conditions were compared with the same tablets coated with Eudragit E™ alone. As shown in Table 4, tablets of the present invention dissolved very homogeneously (RSD<2% after 10 min), while the same tablets coated with only Eudragit E exhibited a higher RSD for longer periods of time (>2% at time points up to 30 min), indicating that they dissolved much less homogeneously.

TABLE 4
Dissolution profiles (as % of API dissolution) of multi-
coated 40 mg atorvastatin (as Atorvastatin Ca) tablets
vs. Eudragit E ™ alone under physiological conditions

| | Coatings | | | |
|---|---|---|---|---|
| | | | 12 mg Eudragit E ™ + 12 mg | |
| | | | Opadry II + 12 mg Acid | |
| | 12 mg Eudragit E ™ | | coat + 12 mg Opadry II | |
| | Average % of | | Average % of | |
| Min | diss. | RSD % | diss. | RSD % |
| 0 | 0 | NA | 0 | NA |
| 5 | 35 | 21.1 | 10 | 59.0 |
| 10 | 78 | 19.6 | 88 | 1.5 |
| 15 | 88 | 9.0 | 94 | 1.5 |
| 20 | 91 | 5.2 | 95 | 1.1 |
| 30 | 95 | 1.7 | 96 | 0.9 |
| 45 | 96 | 1.6 | 98 | 0.8 |

In summary, immediate-release, humidity-resistant atorvastatin dosage forms were developed that exhibited release profiles similar to uncoated tablets, and superior (from the intra-batch homogeneity point of view) to e.g. tablets coated with Eudragit E™ alone, in both acidic and neutral conditions, and probably into the alkaline pH range as well.

### Example 5

### Tablets of the Present Invention (Described in Example 1) are Stable at Elevated Temperatures

### Materials and Experimental Methods

### Method for Detecting Atorvastatin Impurities:

Levels of atorvastatin impurities in the Atorvastatin Calcium 10, 20, and 40 mg tablets were determined by dissolving the powdered tablets in a diluent and analysis by HPLC (gradient method) in a Waters HPLC System by UV Detection. The percentage of impurities of atorvastatin calcium per tablet was calculated by area normalization. Areas that originated from the diluent or placebo and impurity peaks of less than 0.05% were disregarded.

### Method for the Measuring Level of Atorvastatin Calcium:

The content of Atorvastatin Calcium in the 10, 20, and 40 mg tablets was determined by dissolving the powdered tablets in a diluent and analyzing and quantifying it, by comparison with a working standard, by HPLC (isocratic method) in a Waters HPLC System by UV Detection.

### Results

To test the stability of the atorvastatin tablets from Example 1, tablets were packaged in Alu/Alu blisters and placed under conditions of 30° C./65% RH and 40° C./75% RH. RH stands for “relative humidity”.

Before incubation, after a one-month incubation at 40° C. and after a 6 months incubation at 30° C., tablets were examined for appearance of damage or breaks on the coated surface. In addition, tablet weight was determined, the amount of active ingredient was assayed, and levels of impurities were determined. In addition, disintegration time in water and dissolution in neutral medium (Intestinal Fluid, pH 6.8, paddles rotating at 75 rpm) were determined. Previous experience has shown that, if the protective coating is damaged and humidity and/or an acid ingredient reaches the core, atorvastatin Ca is converted into atorvastatin lactone. This would be detected by the assays below as both a decrease in the amount of remaining atorvastatin Ca and a dramatic increase in the level of atorvastatin lactone, thus providing a means of testing the stability of tablets of the present invention.

As shown below in Tables 5-8, tablets of the present invention were stable after 1 month at 40° C./75% RH and 6 months at 30° C./60% RH. Specifically (a) the tablet weight did not increase; (b) the amount of active ingredient did not decrease dramatically (c) the level of impurities (e.g. the Atorvastatin Lactone) increased only slightly, (d) the disintegration time in water did not change significantly; and (e) the dissolution profile remained exactly the same (same delay and same profile). Point (b) demonstrates that the elevated temperature did not degrade the active ingredient. Point (c) is indicative of the advantageous stability of the tablets, since it shows that the integrity of the Eudragit E™ coating was not compromised by the citric acid (if this had not been the case, and a significant amount of citric acid had entered the core, the level of the Lactone impurity would have dramatically increased). Points (d-e) provide further confirmation of the stability of the tablets and more specifically of their coatings to elevated temperature (if the coats and especially the cationic coat had been damaged, the delay at the beginning of the dissolution profile would have disappeared and the disintegration time of the tablets would have decreased).

TABLE 5
Results with 40 mg Atorvastatin (as Atorvastatin
Ca) tablets, coated with the 4 coatings.

| | T = 0 | 1 Month/40° C. | 6 Months/30° C. |
|---|---|---|---|
| Appearance | Conforms to | Conforms to | Conforms to |
| | specifications. | specifications. | specifications. |
| | No cracks on | No cracks on | No cracks on |
| | tablets' coated | coated surface. | coated surface. |
| | surface. | | |
| Weight | 398 | 397 | 398 |
| Assay (mg/tab | 39.29 | 39.37 | 38.87 |
| active ingredient) | | | |
| Impurities | | | |
| Ator. Lactone | 0.07 | 0.21 | 0.49 |
| Max unknown | 0.06 | 0.10 | 0.15 |
| Total | 0.13 | 0.31 | 0.89 |
| Disintegration time | 5′06″ | 4′38″ | 4′46″ |
| in water (min/sec) | | | |
| Dissolution in IF, | Conforms to | Conforms to | Conforms to |
| pH 6.8 | specifications | specifications | specifications |
| 0 min | 0 | 0 | 0 |
| 5 min | 10 | 14 | 23 |
| 10 min | 88 | 89 | 91 |
| 15 min | 94 | 92 | 94 |
| 20 min | 95 | 94 | 95 |
| 30 min | 96 | 95 | 96 |
| 45 min | 98 | 95 | 97 |

TABLE 6
Results with 20 mg atorvastatin (as Atorvastatin Ca)
cores of Example 1 (uncoated cores used for the production
of the 20 mg Atorvastatin coated tablets).

| | T = 0 | 1 Month/40° C. | 6 Months/30° C. |
|---|---|---|---|
| Appearance | Conforms | Conforms | Conforms |
| Weight | 175 | 175 | 175 |
| Assay | 19.99 | 19.52 | 19.36 |
| (mg/tab) | | | |
| Impurities | | | |
| Ator. Lactone | 0.07 | 0.16 | 0.16 |
| Max unknown | 0.07 | 0.07 | 0.14 |
| Total | 0.14 | 0.34 | 0.64 |
| Disintegration | 2′23″ | 1′47″ | 1′37″ |
| time in water | | | |
| Dissolution in | Conforms | Conforms | Conforms |
| IF, pH 6.8 | | | |
| 0 min | 0 | 0 | 0 |
| 5 min | 89 | 90 | 85 |
| 10 min | 95 | 98 | 95 |
| 15 min | 96 | 98 | 96 |
| 20 min | 96 | 99 | 96 |
| 30 min | 97 | 99 | 97 |
| 45 min | 97 | 100 | 97 |

TABLE 7
Results with 20 mg atorvastatin (as Atorvastatin Ca)
tablets of Example 1 (coated with the 4 coatings)

| | T = 0 | 1 Month/40° C. | 6 Months/30° C. |
|---|---|---|---|
| Appearance | Conforms. No | Conforms. No | Conforms. No |
| | cracks present on | cracks present on | cracks present on |
| | tablets' coated | tablets' coated | tablets' coated |
| | surface | surface | surface |
| Weight | 199 | 198 | 198 |
| Assay | 19.69 | 19.93 | 19.34 |
| (mg/tab) | | | |
| Impurities | | | |
| Ator. Lactone | 0.06 | 0.20 | 0.50 |
| Max unknown | 0.08 | 0.12 | 0.18 |
| Total | 0.14 | 0.43 | 0.91 |
| Disintegration | 5′54″ | 4′39 | 4′41″ |
| time in water | | | |
| Dissolution in | Conforms | Conforms | Conforms |
| IF, pH 6.8 | | | |
| 0 min | 0 | 0 | 0 |
| 5 min | 8 | 7 | 4 |
| 10 min | 78 | 88 | 85 |
| 15 min | 90 | 93 | 92 |
| 20 min | 93 | 95 | 94 |
| 30 min | 94 | 96 | 95 |
| 45 min | 95 | 96 | 96 |

TABLE 8
Results with 10 mg atorvastatin (as Atorvastatin Ca)
tablets of Example 1 (coated with the 4 coatings)

| | T = 0 | 1 Month/40° C. | 6 Months/30° C. |
|---|---|---|---|
| Appearance | Conforms. No | Conforms. No | Conforms. No |
| | cracks present on | cracks present on | cracks present on |
| | tablets' coated | tablets' coated | tablets' coated |
| | surface. | surface. | surface. |
| Weight | 99 | 99 | 99 |
| Assay | 9.95 | 9.91 | 9.78 |
| (mg/tab) | | | |
| Impurities | | | |
| Ator. Lactone | 0.06 | 0.16 | 0.32 |
| Max unknown | 0.05 | 0.12 | 0.19 |
| Total | 0.16 | 0.42 | 0.51 |
| Disintegration | 4′28″ | 3′45″ | 3′59″ |
| time in water | | | |
| Dissolution in | Conforms | Conforms | Conforms |
| IF, pH 6.8 | | | |
| 0 min | 0 | 0 | 0 |
| 5 min | 60 | 73 | 58 |
| 10 min | 91 | 93 | 89 |
| 15 min | 94 | 96 | 92 |
| 20 min | 95 | 96 | 93 |
| 30 min | 96 | 98 | 94 |
| 45 min | 97 | 99 | 95 |

In additional experiments, stability studies are conducted using less expensive packaging systems, e.g. PVC/PVDC blisters or plastic bottles, which are less efficient than Alu/Alu blisters in protecting tablets from environmental conditions.

### Example 6

### Production of Additional Atorvastatin Dosage Forms

Additional 40 mg atorvastatin dosage forms were manufactured (containing Atorvastatin as atorvastatin calcium salt in the core), containing some or all of the coatings described in Example 1, with the compositions (in milligrams [mg]) set forth in Table 9.

TABLE 9
Additional atorvastatin dosage forms

| | | Group name | | | | | | |
|---|---|---|---|---|---|---|---|---|
| | | 6.1 | 6.2 | 6.3 | 6.4 | 6.5 | 6.6 | Function |
| Core* | Atorvastatin | 350.00* | 350.00* | 340.00* | | | | Inner |
| | Ca- | | | | | | | immediate |
| | containing | | | | | | | release |
| | core* | | | | | | | core |
| Cationic | Eudragit E | | | 5.0 | | | | Protects |
| coat | | | | | | | | the core |
| Intermediate | Opadry II | | | | 6.0 | | | Protects |
| | (white) | | | | | | | the |
| coat | | | | | | | | cationic |
| | | | | | | | | coat |
| Acid | Citric | | | | | 9.00 | | Acidifying |
| coat | Acid | | | | | | | agent |
| | Povidone | | | | | 0.48 | | Binder |
| | 30 | | | | | | | |
| | Aerosil | | | | | 2.52 | | Glidant |
| Outer | Opadry II | | 10.0 | | | | 15.0 | Taste |
| coat | (white) | | | | | | | masking |
| | TOTAL | 350.0 | 360.0 | 344.0 | 350.0 | 362.0 | 377.0 | |
| | (actual | | | | | | | |
| | weight) | | | | | | | |

*The qualitative and quantitative formulas of the inner cores from each group were very similar to one another.

Thus, the experimental groups were as follows:
6.1—uncoated cores.
6.2—cores coated with 10 mg Opadry II™.
6.3—cores coated with 5 mg Eudragit E™.
6.4—cores coated with 5 mg Eudragit E™ and 6 mg Opadry II™.
6.5—cores coated with 5 mg Eudragit E™, 6 mg Opadry II™, and 13 mg acid coat.
6.6—cores coated with 5 mg Eudragit E™, 6 mg Opadry II™, 13 mg acid coat, and an additional 15 mg Opadry II™.

### Process:

Eudragit E coating solution: 27 g of Eudragit E were dissolved in 423 g Ethanol.

Opadry II coating suspension: 90 g of Opadry II (white) were mixed vigorously for 1H in 573 g purified water USP.

For the coating of the group 6.2 tablets, the same suspension was prepared in a larger amount.

Acid coat coating suspension: 2.16 g Povidone K30 and 40.5 g citric acid anhydrous were dissolved in 22.9 g purified water USP and 186.5 Ethanol. After complete dissolution, 11.34 g Aerosil was added, and the coating suspension was mixed vigorously until the end of the coating process.

Coating process: 467.5 g of uncoated cores were coated successively in a small-scale perforated pan coater successively with one or more, as applicable, of the following coatings: 1) the Eudragit-E coating solution, 2) the Opadry II coating suspension, 3) the acid coat coating suspension, and 4) another layer of the Opadry II coating suspension. The coating process was performed according to the general state of the art.

For the coating of group 6.2 tablets, the batch size was about 7 kg and a larger perforated pan coater apparatus was used.

### Example 7

### Humidity Resistance of Dosage Forms with Various Layers

To determine the humidity resistance of atorvastatin dosage forms with various layers, five cores or tablets of each lot from the previous Example 6 were placed in an open glass bottle. The bottles were placed in an open space, and the weight of the tablets was monitored regularly. As depicted in Tables 10-11, addition of 10 mg of an Opadry II™ coat (group 6.2, whose cores were identical to cores 6.1) did not confer any moisture protection effect. On the other hand, addition of 5 mg Eudragit E™ to the cores (these were very similar to cores 6.1) conferred better but not total moisture protection than 10 mg Opadry II™.

By further addition of an Opadry II™ coating, an acid coat, and another Opadry II™ coating layer (group 6.6), much better moisture protection was achieved, while the release profile of the tablets in slightly acidic or neutral remained fast and homogeneous, with a lag time relative to uncoated tablets of not more than 5 minutes (as shown in results of Example 4 and 5).

TABLE 10
Humidity resistance of dosage forms with various layers

| | Group name | | | | | | | |
|---|---|---|---|---|---|---|---|---|
| | 6.1 | | 6.2 | | | 6.3 | | |
| | Definition | | | | | | | |
| | | | Cores coated with 10 mg | | | Cores coated with 5 mg | | |
| | Uncoated | | Opadry II | | | Eudragit E | | |
| | cores | | | | Water | | | Water |
| Time | Weight | Water | Weight | Water | uptake | Weight | Water | uptake |
| (Hours) | (mg) | uptake | (mg) | uptake | ratio* | (mg) | uptake | ratio* |
| 0 | 1.7409 | 0.00% | 1.7928 | 0.00% | NA | 1.7196 | 0.00% | NA |
| 1 | 1.7451 | 0.24% | 1.7972 | 0.25% | 102% | 1.7226 | 0.17% | 72% |
| 21 | 1.7842 | 2.49% | 1.8413 | 2.71% | 109% | 1.7634 | 2.55% | 102% |
| 24.5 | 1.7851 | 2.54% | 1.8441 | 2.86% | 113% | 1.7664 | 2.72% | 107% |
| 47.5 | 1.7964 | 3.19% | 1.8546 | 3.4% | 108% | 1.7816 | 3.6% | 113% |

*Water uptake ratio is expressed in Tables 10-11 as a percentage of water uptake of uncoated cores.

TABLE 11
Humidity resistance of dosage forms with additional layers

| | Group name | | | | | | | | |
|---|---|---|---|---|---|---|---|---|---|
| | 6.4 | | | 6.5 | | | 6.6 | | |
| | Description | | | | | | | | |
| | 6.3 tablets coated with a | | | 6.4 tablets coated with | | | 6.5 tablets coated with | | |
| | 6 mg Opadry II coat | | | 12 mg acid coat | | | another 15 mg Opadry II | | |
| Time | Weight | Water | | Weight | Water | | Weight | Water | |
| (hours) | (mg) | uptake | ratio* | (mg) | uptake | ratio* | (mg) | uptake | ratio* |
| 0 | 1.7479 | 0.00% | NA | 1.818 | 0.00% | NA | 1.886 | 0.00% | NA |
| 1 | 1.7506 | 0.15% | 64% | 1.8191 | 0.06% | 25% | 1.8869 | 0.05% | 20% |
| 21 | 1.7879 | 2.29% | 92% | 1.8513 | 1.83% | 74% | 1.8999 | 0.74% | 30% |
| 24.5 | 1.7909 | 2.46% | 97% | 1.8548 | 2.02% | 80% | 1.9007 | 0.78% | 31% |
| 47.5 | 1.8073 | 3.4% | 107% | 1.875 | 3.1% | 98% | 1.9189 | 1.7% | 55% |

As shown by the above, tablets of the present invention confer very effective moisture protection without exhibiting a modified dissolution profile (same or at least highly similar dissolution profile and same or at least highly similar homogeneity) relative to uncoated cores, over the entire range of pH conditions from acidic to neutral.

### Example 8

### Production of Highly Humidity-Resistant 20, and 40 mg Atorvastatin Tablets with Higher Amounts of Eudragit-E Cationic Coat but which Still Would Open Quickly Even in Mildly Acidic and Neutral Aqueous Medium

Experiments were conducted to check the applicability of the invention with high amounts of cationic Eudragit E™ coat which would provide a very high humidity protection to the core, which would delay the dissolution profile by only 5 or 10 minutes in pH 1.2, which would hardly dissolve in pH4.5 and pH6.8 without the presence of the acid coat and which would better dissolve in pH4.5 and pH6.8 in presence of the acid coat.

The tablets had the composition of table 12 and were coated according to the same principles as those described in example 1:

TABLE 12
Composition of the 20 and 40 mg atorvastatin (as
Atorvastatin Ca) tablets in milligrams (mg).

| | | Group name | | | |
|---|---|---|---|---|---|
| | | Formula 8.1 | Formula 8.2 | | |
| | | Dosage | | | |
| | | 20 mg | 40 mg | % in the formula | Function |
| Core | Atorvastatin | 175.00 | 350.00 | 83.3% | Inner |
| (mg) | Ca-containing | | | | immediate |
| | core* | | | | release core |
| Subcoat | Opadry II | 5.00 | 10.00 | 2.4% | Protects core |
| (mg) | (white) ™ | | | | from cationic |
| | | | | | coat |
| Cationic | Eudragit-E ™ | 10.00 | 20.00 | 4.8% | Protects core |
| coat | | | | | |
| (mg) | | | | | |
| Intermed. | Opadry tm ™ | 6.00 | 12.00 | 2.9% | Protects |
| coat. | (white)** | | | | cationic coat |
| (mg) | | | | | |
| Acid | Citric Acid | 4.50 | 9.00 | 2.1% | Acidifying |
| coat | | | | | agent |
| (mg) | Povidone 30 | 0.24 | 0.48 | 0.1% | Binder |
| | Aerosil | 1.26 | 2.52 | 0.6% | Glidant |
| Outer | Opadry tm ™ | 8.00 | 16.00 | 3.8% | Taste masking |
| coat | (white)** | | | | |
| (mg) | | | | | |
| | TOTAL (mg) | 210.0 | 420.0 | 100.0% | |

*The qualitative and quantitative formulas and the production process of the cores are very close to those of cores of example 1.
**Opadry tm ™ (white) is another grade of Opadry which still dissolves rapidly in any aqueous medium but provides an improved taste masking effect..

In vitro dissolution studies of the tablets (of table 12) coated only with the 2 first coats [Opadry II (white)™+Eudragit-E™] compared with the tablets coated with the 5 coats [Opadry II (white)™+Eudragit-E™+Opadry tm™ (white)+Acid coat+Opadry tm™ (white)] were performed in neutral conditions similar to conditions in the small intestine, i.e. in IF (phosphate buffer simulating intestinal fluid, pH 6.8) or in mildly acidic conditions Phosphate Buffer pH 4.5 with a paddle rotating at 75 rpm (6 tablets tested for each lot). The results are summarized in the following tables:

TABLE 13
20 mg Atorvatatin coated tablets: Formula
8.1 tested in Phosphate Buffer pH 4.5

| | Coatings | | | |
|---|---|---|---|---|
| | | | 5 mg Opadry II (white) ™ + 10 mg | |
| | 5 mg Opadry II | | Eudragit-E ™ + 6 mg Opadry tm ™ | |
| | (white) ™ + 10 mg | | (white) + 6 mg Acid coat + 8 mg | |
| | Eudragit E ™ | | Opadry tm ™ (white) | |
| | Min and Max | | Min and Max | |
| Min | % of diss. | RSD % | % of diss. | RSD % |
| 0 | 0 | — | 0 | — |
| 5 | 0 | — | 0 | — |
| 10 | 0 | — | 0 | — |
| 15 | 0 | — | 0-49 | 74% |
| 20 | 0 | — | 36-68 | 20% |
| 30 | 1-65 | 59% | 58-79 | 10% |
| 45 | 70-84 | 7% | 69-85 | 7% |

TABLE 14
20 mg Atorvatatin coated tablets: Formula
8.1 tested in IF Phosphate Buffer pH 6.8

| | Coatings | | | |
|---|---|---|---|---|
| | | | 5 mg Opadry II (white) ™ + 10 mg | |
| | 5 mg Opadry II | | Eudragit-E ™ + 6 mg Opadry tm ™ | |
| | (white) ™ + 10 mg | | (white) + 6 mg Acid coat + 8 mg | |
| | Eudragit E ™ | | Opadry tm ™ (white) | |
| | Min and Max | | Min and Max | |
| Min | % of diss. | RSD % | % of diss. | RSD % |
| 0 | 0 | — | 0 | — |
| 5 | 0 | — | 0 | — |
| 10 | 0 | — | 0 | — |
| 15 | 0 | — | 0 | — |
| 20 | 0 | — | 2-10 | 78% |
| 30 | 0 | — | 11-80 | 48% |
| 45 | 0 | — | 58-92 | 19% |

TABLE 15
40 mg Atorvatatin coated tablets: Formula
8.2 tested in Phosphate Buffer pH 4.5

| | Coatings | | | |
|---|---|---|---|---|
| | | | 10 mg Opadry II (white) ™ + 20 mg | |
| | 10 mg Opadry II | | Eudragit E ™ + 12 mg Opadry tm ™ | |
| | (white) ™ + 20 mg | | (white) + 12 mg Acid coat + 16 mg | |
| | Eudragit E ™ | | Opadry tm ™ (white) | |
| | Min and Max | | Min and Max | |
| Min | % of diss. | RSD % | % of diss. | RSD % |
| 0 | 0 | — | 0 | — |
| 5 | 0 | — | 0 | — |
| 10 | 0 | — | 0 | — |
| 15 | 0 | — | 0 | — |
| 20 | 0 | — | 20-38 | 24% |
| 30 | 3-42 | 71% | 49-58 | 6% |
| 45 | 46-63 | 5% | 61-67 | 4% |

TABLE 16
40 mg Atorvatatin coated tablets: Formula
8.2 tested in IF Phosphate Buffer pH 6.8

| | Coatings | | | |
|---|---|---|---|---|
| | | | 10 mg Opadry II (white) ™ + 20 mg | |
| | 10 mg Opadry II | | Eudragit E ™ + 12 mg Opadry tm ™ | |
| | (white) ™ + 20 mg | | (white) + 12 mg Acid coat + 16 mg | |
| | Eudragit E ™ | | Opadry tm ™ (white) | |
| | Min and Max | | Min and Max | |
| Min | % of diss. | RSD % | % of diss. | RSD % |
| 0 | 0 | — | 0 | — |
| 5 | 0 | — | 0 | — |
| 10 | 0 | — | 0 | — |
| 15 | 0 | — | 0 | — |
| 20 | 0 | — | 0 | — |
| 30 | 0 | — | 2-47 | 66% |
| 45 | 0 | — | 60-90 | 18% |

Results clearly show that the presence of the acid coat on the tablets very significantly speeds up the opening of the Eudragit-E coat and improves the homogeneity of the dissolution profile of the tablets both at pH4.5 and pH6.8. This means that the addition of the acid coat would increase the probability, speed up and improve the homogeneity of the in vivo opening of the tablets in certain conditions such as:
fed conditions (in which the pH of the stomach can increase up to ˜pH5) which would decrease the bad influence of the food effect on the pharmacokinetics of the product
in case the tablet is expelled too quickly from the stomach to the duodenum whose pH is typically close to 6.8.

### Example 9

### Long Term Stability of a Coated Formula of the Invention

A formula of the invention described in Table 17 was produced according to a method similar as that described in example 1. Then the formula was challenged in a long term stability program. The main goal of the experiment was to test the stability of the coating system of the invention for a long period both in regular and stress conditions.

TABLE 17
Composition of the 40 mg atorvastatin (as Atorvastatin Ca) tablets in milligrams (mg).

| | | Group name | | | |
|---|---|---|---|---|---|
| | | Formula 9.1 | Formula 9.2 | | |
| | | Dosage | | | |
| | | 40 mg dosage uncoated | 40 mg dosage coated | % in the formula | Function |
| Core | Atorvastatin | 300.00 | | 88.2% | Inner |
| (mg) | Ca-containing | | | | immediate |
| | core* | | | | release core |
| Cationic | Eudragit E ™ | — | 20.00 | 5.9% | Protects core |
| coat | | | | | |
| (mg) | | | | | |
| Acid | Citric Acid | — | 7.50 | 2.2% | Acidifying |
| coat | | | | | agent |
| (mg) | Povidone 30 | — | 0.40 | 0.1% | Binder |
| | Aerosil | — | 2.10 | 0.6% | Glidant |
| Cationic | Eudragit E ™ | — | 10.00 | 2.9% | Taste masking |
| coat | | | | | |
| (mg) | | | | | |
| | TOTAL (mg) | 300.0 | 340.0 | 100.0% | |

*The qualitative and quantitative formulas and the production process of the cores were very close to those of cores of example 1.

To test the stability of the coating system of the invention, the uncoated 9.1 and the coated tablets 9.2 formulas from table 17 were packaged in Alu/Alu blisters and incubated under conditions of 25° C./60% RH, 30° C./65% RH and 40° C./75% RH for comparison.

Before incubation and during the stability program, tablets were examined for general appearance and damage or breaks on the coated surface. In addition, tablet weight was determined, the amount of active ingredient was assayed, and levels of impurities were determined. In addition, disintegration time in HCl 0.1N and dissolution in neutral medium (45 min in Intestinal Fluid, pH 6.8, paddles rotating at 75 rpm for formula 9.1) and (10 min in 750 ml HCl 0.1N followed by 35 min in Intestinal Fluid, pH 6.8, paddles rotating at 75 rpm for formula 9.2) were determined. Previous experience has shown that, if the protective coating is damaged and humidity and/or an acid ingredient reaches the core, atorvastatin Ca would convert into atorvastatin lactone. This would be detected by the assays below as both a decrease in the amount of remaining atorvastatin Ca and an increase in the level of atorvastatin lactone, thus providing a means of testing the stability of the coating system and tablets of the present invention.

As shown below in Tables 18-20, tablets of formula 9.1 and 9.2 were stable after 6 months at 40° C./75% RH and after 12 months at 30° C./65% RH and 25° C./60% RH. Specifically (a) the tablets weight did not increase; (b) the amount of active ingredient did not decrease more in formula 9.2 than in formula 9.1; (c) the level of impurities (especially the Atorvastatin Lactone) increased only slightly and did not increase more in formula 9.2 than in formula 9.1; (d) the disintegration time in HCl did not change significantly neither in formula 9.1 nor in formula 9.2; and (e) the dissolution profile remained unchanged both in formula 9.1 and in formula 9.2 (same delay and same profile). Point (b) demonstrates that the elevated temperature did not degrade the active ingredient. Points (b) and (c) are indicative of the advantageous stability of the tablets, since it shows that the integrity of the Eudragit E™ coating was not compromised by the citric acid (if a lot of citric acid had entered the core, the level of the Lactone impurity would have increased more in formula 9.2 than in formula 9.1). Points (d-e) provide further confirmation of the stability of the tablets and more specifically of their coatings to elevated temperature (if the coats and especially the cationic coat had been damaged, the delay at the beginning of the dissolution profile would have disappeared and the disintegration time of the coated tablets 9.2 would have decreased).

TABLE 18
Stability results with 40 mg Atorvastatin uncoated
and coated tablets of table 17 at 25° C./60% RH.

| | Uncoated cores of table 17 | | Coated tablets of table 17 | |
|---|---|---|---|---|
| | (formula 9.1) | | (formula 9.2 coated with 5 coating layers) | |
| | T = 0 | 12 Months/25° C. | T = 0 | 12 Months/25° C. |
| Appearance | Conforms to | Conforms to | Conforms to | Conforms to |
| | specifications. | specifications. | specifications. | specifications. |
| | | | No cracks on | No cracks on |
| | | | coated surface. | coated surface. |
| Weight (mg) | 301 | 301 | 341 | 339 |
| Assay (mg/tab | 39.99 | 39.08 | 39.63 | 39.40 |
| active ingredient) | | | | |
| Impurities | | | | |
| Ator. Lactone | 0.10 | 0.19 | 0.09 | 0.17 |
| Max unknown | 0.05 | 0.16 | 0.08 | 0.19 |
| Total | 0.15 | 0.43 | 0.17 | 0.50 |
| Disintegration | 4′22″ | 4′41″ | 6′48″ | 7′12″ |
| time in HCl 0.1N | | (at 3 months) | | (at 3 months) |
| (min′ sec″) | | | | |
| Dissolution in IF, | Conforms to | Conforms to | Conforms to | Conforms to |
| pH 6.8 | specifications | specifications | specifications | specifications |
| | | (at 9 months) | | (at 3 months) |
| 0 min | 0 | 0 | 0 | 0 |
| 5 min | 70 | 62 | — | — |
| 10 min | 92 | 90 | — | — |
| 15 min | 95 | 91 | 73 | 76 |
| 20 min | — | 92 | 84 | 81 |
| 30 min | 97 | 94 | 92 | 84 |
| 45 min | — | 95 | 95 | 87 |

TABLE 19
Stability results with 40 mg Atorvastatin uncoated
and coated tablets of table 17 at 30° C./65% RH.

| | Uncoated cores of table 17 | | Coated tablets of table 17 | |
|---|---|---|---|---|
| | (formula 9.1) | | (formula 9.2 coated with 5 coating layers) | |
| | T = 0 | 12 Months/30° C. | T = 0 | 12 Months/30° C. |
| Appearance | Conforms to | Conforms to | Conforms to | Conforms to |
| | specifications. | specifications. | specifications. | specifications. |
| | | | No cracks on | No cracks on |
| | | | coated surface. | coated surface. |
| Weight (mg) | 301 | 301 | 341 | 340 |
| Assay (mg/tab | 39.99 | 39.47 | 39.63 | 39.30 |
| active ingredient) | | | | |
| Impurities | | | | |
| Ator. Lactone | 0.10 | 0.25 | 0.09 | 0.23 |
| Max unknown | 0.05 | 0.20 | 0.08 | 0.22 |
| Total | 0.15 | 0.79 | 0.17 | 0.70 |
| Disintegration | 4′22″ | 4′27″ | 6′48″ | 7′24″ |
| time in HCl 0.1N | | (at 3 months) | | (at 3 months) |
| (min/sec) | | | | |
| Dissolution in IF, | Conforms to | Conforms to | Conforms to | Conforms to |
| pH 6.8 | specifications | specifications | specifications | specifications |
| | | (at 9 months) | | (at 3 months) |
| 0 min | 0 | 0 | 0 | 0 |
| 5 min | 70 | 61 | — | — |
| 10 min | 92 | 90 | — | — |
| 15 min | 95 | 92 | 73 | 81 |
| 20 min | — | 93 | 84 | 87 |
| 30 min | 97 | 94 | 92 | 90 |
| 45 min | — | 96 | 95 | 93 |

TABLE 20
Stability results with 40 mg Atorvastatin uncoated
and coated tablets of table 17 at 40° C./75% RH.

| | Uncoated cores of table 17 | | Coated tablets of table 17 | |
|---|---|---|---|---|
| | (formula 9.1) | | (formula 9.2 coated with 5 coating layers) | |
| | T = 0 | 6 Months/40° C. | T = 0 | 6 Months/40° C. |
| Appearance | Conforms to | Conforms to | Conforms to | Conforms to |
| | specifications. | specifications. | specifications. | specifications. |
| | | | No cracks on | No cracks on |
| | | | coated surface. | coated surface. |
| Weight (mg) | 301 | 301 | 341 | 339 |
| Assay (mg/tab | 39.99 | 38.59 | 39.63 | 38.10 |
| active ingredient) | | | | |
| Impurities | | | | |
| Ator. Lactone | 0.10 | 0.36 | 0.09 | 0.33 |
| Max unknown | 0.05 | 0.22 | 0.08 | 0.26 |
| Total | 0.15 | 1.10 | 0.17 | 1.12 |
| Disintegration | 4′22″ | 4′34″ | 6′48″ | 7′11″ |
| time in HCl 0.1N | | (at 3 months) | | (at 3 months) |
| (min/sec) | | | | |
| Dissolution in IF, | Conforms to | Conforms to | Conforms to | Conforms to |
| pH 6.8 | specifications | specifications | specifications | specifications |
| | | (at 3 months) | | (at 3 months) |
| 0 min | 0 | NP | 0 | 0 |
| 5 min | 70 | NP | — | — |
| 10 min | 92 | NP | — | — |
| 15 min | 95 | NP | 73 | 84 |
| 20 min | — | NP | 84 | 88 |
| 30 min | 97 | NP | 92 | 91 |
| 45 min | — | NP | 95 | 93 |

In additional experiments, stability studies will be conducted using less expensive packaging systems, e.g. PVC/PVDC blisters or plastic bottles, which are less efficient than Alu/Alu blisters in protecting tablets from environmental conditions.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1.-52. (canceled)

53. A pharmaceutical composition, comprising: (i) a humidity-sensitive core comprising an active ingredient or pharmaceutically acceptable salt thereof; (ii) a coating over the core, the coating containing a cationic polymer; and (iii) an additional coating over the cationic polymer-containing coating, the additional coating comprising an acidifying agent.

54. The pharmaceutical composition of claim 53, wherein the cationic polymer is selected from the group consisting of a cationic polyamine, a cationic polyacrylamide, a cationic polyethyleneimine, a gelatin, a polyvinyl-containing compound, a polyvinylimidazole, a polyamidine-containing copolymer, a cationic starch, a cationic polysaccharide, an ammonium chloride-containing polymer, an epichlorohydrin-containing compound, an N-vinyl polymer, and a salt of any of the above.

55. The pharmaceutical composition of claim 54, wherein the cationic polymer is a methacrylate polymer, or wherein the cationic polymer comprises monomers of an amine methacrylate, a methacrylic ester, or a combination thereof.

56. The pharmaceutical composition of claim 53, wherein the acidifying agent is selected from the group consisting of citric acid, N-acetylglutamic acid, adipic acid, aldaric acid, alpha-ketoglutaric acid, aspartic acid, azelaic acid, camphoric acid, creatine-alpha ketoglutarate, diglycolic acid, dimercaptosuccinic acid, fumaric acid, glutaconic acid, glutamic acid, glutaric acid, isophthalic acid, itaconic acid, maleic acid, malic acid, malonic acid, meglutol, mesaconic acid, mesoxalic acid, 3-methylglutaconic acid, muconic acid, oxalic acid, oxaloacetic acid, pamoic acid, phthalic acids, pimelic acid, sebacic acid, suberic acid, succinic acid, tartaric acid, tartronic acid, terephthalic acid, traumatic acid, methanoic acid, ethanoic acid, propanoic acid, butanoic acid, pentanoic acid, hexanoic acid, heptanoic acid, octanoic acid, nonanoic acid, decanoic acid, dodecanoic acid, hexadecanoic acid, octadecanoic acid, acrylic acid, a fatty acid, docosahexaenoic acid, eicosapentaenoic acid, aspartic acid, glutamic acid, a keto acid, an aromatic carboxylic acid, pyruvic acid, acetoacetic acid, benzoic acid, salicylic acid, dicarboxylic acids, tricarboxylic acids, alpha hydroxy acids, lactic acid, propionic acid, glycolic acid, cinnamic acid, mandelic acid, methanesulfonic acid, ethanesulfonic acid, p-toluenesulfonic acid, and palmatic acid.

57. The pharmaceutical composition of claim 53, wherein the additional coating further comprises a binder, wherein the binder is a water-soluble polymer.

58. The pharmaceutical composition of claim 53, wherein the pharmaceutical composition further comprises at least one intermediate coating between the cationic polymer containing coating and the additional coating, wherein the intermediate coating is water-soluble or readily disintegrates in aqueous solution.

59. The pharmaceutical composition of claim 53, wherein the pharmaceutical composition further comprises at least one outer coating over the additional coating, wherein the outer coating is water-soluble or readily disintegrates in aqueous solution, or wherein the outer coating comprises a water-soluble polymer, a taste-masking agent, or a combination thereof.

60. The pharmaceutical composition of claim 53, wherein the pharmaceutical composition is an immediate release pharmaceutical composition.

61. The pharmaceutical composition of claim 53, wherein the active ingredient is a statin or a pharmaceutically acceptable salt thereof.

62. The pharmaceutical composition of claim 61, wherein the statin is atorvastatin or a pharmaceutically acceptable salt thereof.

63. The pharmaceutical composition of claim 61, wherein the pharmaceutically acceptable salt is atorvastatin calcium.

64. The pharmaceutical composition of claim 61, wherein the statin is present in the pharmaceutical composition in a humidity-sensitive form.

65. The pharmaceutical composition of claim 53, wherein the core comprises a major excipient, a minor excipient or a combination thereof, wherein the major excipient is selected from the group consisting of starch, pregelatinized starch or lactose, and a combination thereof, and wherein the minor excipient is selected from the group consisting of HPC, HPMC, PVP, Crospovidone, Tween, Magnesium stearate, Aerosil, and a combination thereof.

66. The pharmaceutical composition of claim 53, wherein said pharmaceutical composition comprises a humidity-sensitive active ingredient, a humidity-sensitive excipient, or a combination thereof.

67. The pharmaceutical composition of claim 53, wherein the core further comprises an additional coating between the core and the cationic polymer containing coating, wherein the additional coating is a protecting or a functional coating, and comprises a sugar coat, a polymer coat, an enteric-coat, a delayed release coat, or a modified release coat.

68. A method for lowering the cholesterol level of a subject in need thereof, the method comprising the step of administering to the subject the pharmaceutical composition of claim 53, thereby lowering the cholesterol level of a subject.

69. A method for preparation of a pharmaceutical composition comprising an active ingredient or a pharmaceutically acceptable salt thereof, comprising the steps of: (a) applying a cationic polymer containing coating over a humidity-sensitive core comprising the active ingredient or pharmaceutically acceptable salt thereof; and (b) applying an additional coating over the cationic polymer-containing coating, wherein the additional coating comprises an acidifying agent.

70. The method of claim 69, wherein the cationic polymer is selected from the group consisting of a cationic polyamine, a cationic polyacrylamide, a cationic polyethyleneimine, a gelatin, a polyvinyl-containing compound, a polyvinylimidazole, a polyamidine-containing copolymer, a cationic starch, a cationic polysaccharide, an ammonium chloride-containing polymer, a epichlorohydrin-containing compound, an N-vinyl polymer, and a salt of any of the above.

71. The method of claim 70, wherein the cationic polymer is a methacrylate polymer, or wherein the cationic polymer comprises monomers of an amine methacrylate, a methacrylic ester, or a combination thereof.

72. The method of claim 69, wherein the acidifying agent is selected from the group consisting of citric acid, N-acetylglutamic acid, adipic acid, aldaric acid, alpha-ketoglutaric acid, aspartic acid, azelaic acid, camphoric acid, creatine-alpha ketoglutarate, diglycolic acid, dimercaptosuccinic acid, fumaric acid, glutaconic acid, glutamic acid, glutaric acid, isophthalic acid, itaconic acid, maleic acid, malic acid, malonic acid, meglutol, mesaconic acid, mesoxalic acid, 3-methylglutaconic acid, muconic acid, oxalic acid, oxaloacetic acid, pamoic acid, phthalic acids, pimelic acid, sebacic acid, suberic acid, succinic acid, tartaric acid, tartronic acid, terephthalic acid, traumatic acid, methanoic acid, ethanoic acid, propanoic acid, butanoic acid, pentanoic acid, hexanoic acid, heptanoic acid, octanoic acid, nonanoic acid, decanoic acid, dodecanoic acid, hexadecanoic acid, octadecanoic acid, acrylic acid, a fatty acid, docosahexaenoic acid, eicosapentaenoic acid, aspartic acid, glutamic acid, a keto acid, an aromatic carboxylic acid, pyruvic acid, acetoacetic acid, benzoic acid, salicylic acid, dicarboxylic acids, tricarboxylic acids, alpha hydroxy acids, lactic acid, propionic acid, glycolic acid, cinnamic acid, mandelic acid, methanesulfonic acid, ethanesulfonic acid, p-toluenesulfonic acid, and palmatic acid.

73. The method of claim 69, wherein the additional coating further comprises a binder, wherein the binder is a water-soluble polymer.

74. The method of claim 69, further comprising the step of applying one or more additional coating between the core and the cationic polymer containing coating, wherein the additional coating is a protecting or a functional coating, and comprises a sugar coat, a polymer coat, an enteric-coat, a delayed release coat, or a modified release coat.

75. The method of claim 69, further comprising the step of applying at least one intermediate coating between the cationic polymer containing coating and the additional coating, wherein the intermediate coating is water-soluble or readily disintegrates in aqueous solution.

76. The method of claim 69, further comprising the step of applying at least one outer coating over the additional coating, wherein the outer coating is water-soluble or readily disintegrates in aqueous solution, or wherein the outer coating comprises a water-soluble polymer, a taste-masking agent, or a combination thereof.

77. The method of claim 69, wherein the pharmaceutical composition is an immediate release pharmaceutical composition.

78. The method of claim 69, wherein the active ingredient is a statin or a pharmaceutically acceptable salt thereof.

79. The method of claim 78, wherein the statin is present in the pharmaceutical composition in a humidity-sensitive form.

80. The method of claim 69, wherein said pharmaceutical composition comprises a humidity-sensitive active ingredient, a humidity-sensitive excipient, or a combination thereof.
